(19) United States
(12) Patent Application Publication
(10) Pub. No.: US 2023/0399405 A1
(43) Pub. Date: Dec. 14, 2023

(54) VISTA AGONIST FOR TREATMENT/PREVENTION OF ISCHEMIC AND/OR REPERFUSION INJURY

(71) Applicant: THE TRUSTEES OF DARTMOUTH COLLEGE, Hanover, NH (US)

(72) Inventors: Randolph J. NOELLE, Plainfield, NH (US); Nicole SMITS, Hanover, NH (US)

(21) Appl. No.: 18/035,399

(22) PCT Filed: Nov. 4, 2021
(86) PCT No.: PCT/US2021/058020

§ 371 Date: May 4, 2023

Related U.S. Application Data
(60) Provisional application No. 63/109,584, filed on Nov. 4, 2020.

(51) Int. Cl. C07K 16/28; A61P 9/10; C07K 14/705; A61P 13/12

(52) U.S. Cl. CPC C07K 16/2827; C07K 16/283; C07K 16/2896; A61P 9/10; C07K 14/70532; C07K 14/70596; A61P 13/12; C07K 2319/30; C07K 2317/565

(57) ABSTRACT

This invention provides methods for treating and preventing injury caused by ischemia followed by reperfusion by the administration of VISTA agonist, optionally an agonist anti-VISTA antibody or VISTA fusion protein. This invention specifically relates to the treatment and prevention of ischemic reperfusion injury HRH and conditions associated therewith including myocardial infarction, cardiac surgery, stroke, solid organ transplant recipients and post-surgery acute kidney injury by the administration of an agonist anti-VISTA antibody.

### RELATED APPLICATIONS

This invention provides claims priority to U.S. Provisional Application No. 63/109,584, filed on Nov. 4, 2020, the contents of which are incorporated by reference in their entirety.

### FIELD

This invention provides new therapies for treating or preventing ischemic reperfusion injury, conditions associated therewith and methods for treating patients particularly susceptible thereto, e.g., patients exhibiting or at risk of developing myocardial infarction, cardiac surgery patients, patients exhibiting or at risk of stroke, solid organ transplant recipients, particularly those receiving organs from deceased donors, and patients with post-surgery acute kidney injury.

### BACKGROUND

This invention relates to the treatment and prevention of ischemic reperfusion injury (IRI) and conditions associated therewith including myocardial infarction, cardiac surgery, stroke, solid organ transplant recipients and post-surgery acute kidney injury.

IRI occurs following blood flow restoration in a tissue that suffered an anoxic event. Ischemic events result in oxygen and nutrient deprivation to tissues, resulting in metabolic alterations and an accumulation of waste products. Disruptions in ion homeostasis contribute to cell/tissue death through apoptosis and necrosis. Restoration of blood flow triggers additional tissue damage through a sudden increase in oxygen radicals.

IRI can occur throughout the body and can result from various trauma, including: myocardial infarction, cardiac surgery, stroke and solid organ transplant. Presently, there are no FDA-approved therapies for the treatment of IRI.

Patients experiencing STEMI myocardial infarctions are at the highest risk for IRI. Acute myocardial infarction (AMI) is a leading cause of morbidity and mortality worldwide. Timely reperfusion following AMI through either thrombolytic therapy or primary percutaneous coronary intervention is essential to limit infarct size¹. However, rapid reperfusion increases the risk of myocardial IRI—resulting in reperfusion-induced arrhythmias. IRI is most common in patients presenting with acute ST-elevation myocardial infarction (STEMI) since the most effective therapeutic option is timely reperfusion. STEMI patients suffer a complete blockage in the coronary artery, resulting in cardiac muscle injury and death. Approximately 790,000 patients in the US and 1.685M patients in the EU5 experience AMI annually, of which ˜20% are considered STEMI^{2,3,4}. Approximately 495,000 patients in the US and EU⁴ suffer from STEMI annually.

Acute kidney injury (AKI) may occur following cardiac surgery (Cardiac Surgery Associated Acute Kidney Injury (CSA-AKI)). Post-surgical AKI develops within hours to days following ischemic conditions caused by reduced blood flow to the kidneys during surgery. Patients undergoing open chest cardiovascular surgery with the use of cardiopulmonary bypass (CPB) are susceptible to IRI. In 2018, approximately 140,000 patients in the US underwent cardiac surgeries involving isolated coronary artery bypass grafting (CABP) (120,000 patients) or valve replacement involving CABP (20,000 patients)⁵. In 2014, approximately 122,000 patients in the EU5 underwent cardiac surgeries involving CABP⁶. The rate of AKI development in most patients undergoing cardiac surgery is low, but can be as high as 22-39% in high risk patients⁷. Over 60% of cardiac surgery patients have a moderate to high risk of AKI. Approximately 157,000 patients in the US and EU5 are at risk of developing CSA-AKI. CSA-AKI doubles morbidity and mortality in patients and severe cases can result in mortality rates up to 50%^{7,8}. No specific treatments are available for AKI—current treatment options include hospitalization and dialysis.

Patients suffering ischemic strokes are susceptible to IRI following reperfusion after administration of tPA. Approximately 795,000 patients in the US and 300,000 patients in the EU5 experience a stroke annually^{9,10}. Stroke prevalence is increasing substantially in the US and is estimated to increase by 20.5% from 2012-2030. By 2030, an additional 3.4 million US adults will have had a stroke. Approximately 87% of strokes are ischemic⁹. Approximately 955,000 patients in the US and EU5 experience an ischemic stroke annually. The only FDA-approved drug available for the treatment of ischemic stroke is the anti-clotting factor, recombinant tissue plasminogen activator (tPA). However, tPA must be administered to the patient within 3-4 hours from the onset of the stroke due to risk of intracranial hemorrhage. Based thereon <30% of patients receive treatment with tPA due to late presentation (>3 hours) for care¹¹. Timely administration of tPA significantly improves patient outcomes through reperfusion, but unfortunately can cause additional injury through IRI.

Reperfusion injury is particularly problematic in recipients who receive deceased donor transplants which may reduce graft survival, prolongs hospitalization and requires dialysis. Delayed graft function (DGF) occurs due to acute kidney injury that occurs in the first week following transplantation. Patients undergoing kidney transplant from deceased donors have a higher risk for developing DGF¹². Living donors are intensively screened for renal function and lack of comorbidities. Brain death induces an intense proinflammatory state, which can impact kidney function following transplantation¹³. Reperfusion following transplant contributes to tissue damage. DGF affects approximately 31% of transplanted kidneys from deceased donors¹⁴. Factors contributing to DGF include the length of cold ischemia as well as the extent of warm ischemic injury. Also, susceptible patients are increasing because Kidney transplants performed with kidneys from deceased donors in 2017¹⁵ comprised 15,000 kidney transplants in the US and 12,000 kidney transplants in the EU¹⁵.

Therefore, based on the forgoing, methods for treating and preventing ischemic reperfusion injury and conditions associated therewith are desperately needed; and more particularly effective methods of treating patients particularly susceptible thereto, e.g., patients exhibiting or at risk of developing myocardial infarction, cardiac surgery patients, patients exhibiting signs of or at risk of stroke, solid organ transplant recipients, particularly those receiving organs from deceased donors and/or exhibiting decreased graft function (DGF), and patients exhibiting or at risk of post-surgery acute kidney injury.

### EXEMPLARY EMBODIMENTS

In one exemplary embodiment the invention provides methods of treating and/or preventing ischemic reperfusion injury (IRI) and/or adverse side effects associated with IRI in a subject in need thereof by administering a therapeutically or prophylactically effective amount of a VISTA agonist.

In some exemplary embodiments, the subject is or has received a solid organ transplant, optionally from a deceased donor, and the VISTA agonist is administered prior, during or after transplant, optionally wherein the solid organ is optionally selected from a kidney, liver, heart, lung, intestine, and aorta.

In any of the foregoing exemplary embodiments, the administration of the VISA agonist, optionally an agonist anti-VISTA antibody, prior or after IR injury, prevents or mitigates the effects of solid organ injury, e.g., kidney injury and protects the solid organ, e.g., a kidney.

In any of the foregoing exemplary embodiments the subject has or is to receive a solid organ transplant and the treatment prevents or ameliorates delayed graft function (DGF).

In any of the foregoing exemplary embodiments the VISTA agonist prevents or treats an injury to an organ caused by ischemia, followed by reperfusion.

In any of the foregoing exemplary embodiments the VISTA agonist prevents or treats IRI caused by one or more of the following: surgery, optionally surgery involving major organs, including but not limited to, the kidney, liver, heart, lung, intestine, and aorta, coronary bypass, major vascular repair, liver resection, and transplantation of one or more of the kidney, liver, heart, lung and aorta.

In any of the foregoing exemplary embodiments the VISTA agonist prevents or treats IRI caused by cardiopulmonary bypass during surgery, stroke, liver ischemia, kidney ischemia, aortic occlusion, myocardial occlusion, cardiac arrest, shock and trauma.

In any of the foregoing exemplary embodiments the VISTA agonist prevents or treats IRI caused by ischemic reperfusion injury (IRI) associated with myocardial infarction, cardiac surgery, stroke, solid organ transplant, post-surgery acute kidney injury, cardiopulmonary bypass surgery, liver ischemia, kidney ischemia, aortic occlusion, myocardial occlusion, cardiac arrest, shock and trauma.

In any of the foregoing exemplary embodiments the VISTA agonist is an agonist anti-VISTA antibody which prevents or treats IRI caused by ischemia followed by reperfusion caused by the return of blood supply to a tissue (reperfusion) after a period of lack of oxygen (ischemia).

In any of the foregoing exemplary embodiments the VISTA agonist, optionally an agonist anti-VISTA antibody, prevents or treats IRI caused by post-operative organ dysfunction due to IR injury, e.g., in a subject undergoing a major surgical procedure, optionally cardiac surgery, liver transplantation, liver resection, renal transplantation, lung transplantation, aortic surgery, or major vascular repair.

In any of the foregoing exemplary embodiments the VISTA agonist, optionally an agonist anti-VISTA antibody, prevents or treats remote organ injury associated with IRI, optionally in a solid organ transplant recipient, further optionally a kidney transplant recipient who develops kidney injury due to renal IR and further develops one or more of liver, intestine, and lung dysfunction and/or an inflammatory state suggesting the onset of sepsis.

In any of the foregoing exemplary embodiments, the VISTA agonist, optionally an agonist anti-VISTA antibody, prevents or treats acute kidney injury (AKI), optionally associated with cardiac surgery.

In any of the foregoing exemplary embodiments, the VISTA agonist, optionally an agonist anti-VISTA antibody, and prevents or treats ischemic AKI associated with a major surgical procedure, optionally one involving the kidney, liver, heart or aorta.

In any of the foregoing exemplary embodiments, the VISTA agonist, optionally an agonist anti-VISTA antibody, prevents or treats IRI associated multi-organ dysfunction and/or systemic inflammation.

In any of the foregoing exemplary embodiments, the VISTA agonist, optionally an agonist anti-VISTA antibody, is administered prior, during and/or after a major surgery and protects against IR injury.

In any of the foregoing exemplary embodiments, the VISTA agonist, optionally an agonist anti-VISTA antibody, is administered prior, during and/or after a surgery involving major organs, including but not limited to, the kidney, liver, heart, lung, intestine, and aorta, coronary bypass, major vascular repair, liver resection, and transplantation of the kidney, liver, and lung and protects against IRI injury.

In any of the foregoing exemplary embodiments, the VISTA agonist, optionally an agonist anti-VISTA antibody, is administered prior to a surgery including cardiopulmonary bypass.

In any of the foregoing exemplary embodiments, the VISTA agonist, optionally an agonist anti-VISTA antibody, is administered to a subject diagnosed with or showing signs of stroke, liver ischemia, kidney ischemia, aortic occlusion, myocardial occlusion, cardiac arrest, shock or trauma and such administration protects against and/or inhibits further IR injury.

In any of the foregoing exemplary embodiments, the administered VISTA agonist comprises a VISTA fusion protein, e.g., a VISTA-Ig fusion protein or comprises an agonistic anti-VISTA antibody or antibody fragment.

In any of the foregoing exemplary embodiments, the administered VISTA agonist comprises a human VISTA fusion protein, e.g., a human VISTA-Ig fusion protein or an agonistic anti-human VISTA antibody or antibody fragment.

In any of the foregoing exemplary embodiments, the administered VISTA agonist comprises an agonistic anti-VISTA or antibody fragment which comprises a variable light and heavy chain polypeptide comprising the CDRs of any one of the anti-human VISTA antibodies having the sequences contained in the table in FIG. 6.

In any of the foregoing exemplary embodiments, the administered VISTA agonist comprises an agonistic anti-VISTA or antibody fragment which comprises the variable light and heavy chain polypeptides of any one of the anti-human VISTA antibodies having the sequences contained in the table in FIG. 6.

In any of the foregoing exemplary embodiments, the administered VISTA agonist is used to treat or prevent IRI in any condition that comprises blood flow restoration in a tissue that suffered an anoxic event and ischemic events that result in oxygen and nutrient deprivation to tissues, and metabolic alterations and an accumulation of waste products, optionally myocardial infarction, cardiac surgery, stroke or solid organ transplant.

In any of the foregoing exemplary embodiments, the administered VISTA agonist is used to treat or prevent IRI in patients experiencing STEMI myocardial infarctions.

In any of the foregoing exemplary embodiments, the administered VISTA agonist is used to treat or prevent IRI in patients with or at risk of Acute kidney injury (AKI) following cardiac surgery (Cardiac Surgery Associated Acute Kidney Injury (CSA-AKI)).

In any of the foregoing exemplary embodiments, the administered VISTA agonist is used to treat or prevent IRI in patients showing signs of stroke or at risk of stroke, particularly a patient suffering ischemic strokes susceptible to IRI following reperfusion after administration of tPA.

In any of the foregoing exemplary embodiments, the administered VISTA agonist is used to treat or prevent IRI in transplant recipients, particularly solid organ transplants, and more particularly in patients who receive deceased donor transplants and/or those exhibiting Delayed graft function (DGF) and this promotes graft survival.

In any of the foregoing exemplary embodiments, the VISTA agonist comprises an agonist anti-VISTA antibody or agonistic anti-VISTA antibody fragment or an agonistic VSIG3 fusion protein or an agonistic anti-VSIG3 antibody or agonistic anti-VSIG3 antibody fragment or an agonistic PSGL1 antibody, antibody fragment or PSGL1 fusion protein.

In any of the foregoing exemplary embodiments, the VISTA agonist decreases the levels of at least one of LPS-induced IL-12p40, IL-6, CXCL2 and TNF.

In any of the foregoing exemplary embodiments, the VISTA agonist increases the expression of mediators involved in macrophage tolerance induction, wherein said mediators optionally include at least one of IRG1, miR221, A20, and IL-10 and/or increases the expression of anti-inflammatory transcription factors which drive an anti-inflammatory profile optionally including at least one of IRF5, IRF8, and NFKB1.

In any of the foregoing exemplary embodiments, the VISTA agonist (i) reduces the level of CXCR2 and/or CXCL10; (ii) reduces neutrophil/lymphocyte ratios, (iii) reduces FcgRIII levels or a combination of the foregoing.

In any of the foregoing exemplary embodiments, the VISTA agonist increases the expression of mediators involved in macrophage tolerance induction, wherein said mediators optionally include at least one of IRG1, miR221, A20, and IL-10 and/or increases the expression of anti-inflammatory transcription factors which drive an anti-inflammatory profile optionally including at least one of IRF5, IRF8, and NFKB1.

In any of the foregoing exemplary embodiments, the method also includes the administration of another active, optionally selected from a PD-1 agonist, a CTLA-4 agonist, a TNF antagonist optionally an anti-TNF antibody or TNF-receptor fusion such as Embrel, an IL-6 antagonist such as an anti-IL-6 or anti-IL-6R antibody, a corticosteroid or other anti-inflammatory agent.

In any of the foregoing exemplary embodiments, wherein the agonistic anti-VISTA antibody or antibody fragment specifically binds to human VISTA, optionally an agonistic anti-VISTA antibody or antibody fragment that comprises a variable light chain and a variable heavy chain polypeptide comprising the same CDRs any one of the antibodies having the sequences contained in the table in FIG. 6 or an agonistic anti-VISTA antibody or antibody fragment that comprises a variable light chain and a variable heavy chain polypeptide comprising the same CDRs any one of the antibodies having the sequences contained in the table in FIG. 6 and the variable light chain and the variable heavy chain polypeptide of said antibody or antibody fragment respectively each possess at least 90% sequence identity to the variable light chain and the variable heavy chain polypeptides of the same anti-human VISTA antibody having the sequences contained in the table in FIG. 6.

In any of the foregoing exemplary embodiments, the agonistic anti-VISTA antibody or antibody fragment comprises a variable light chain and a variable heavy chain polypeptide comprising the same sequences as any one of the anti-human VISTA antibodies having the sequences contained in the table in FIG. 6.

In any of the foregoing exemplary embodiments, the VISTA agonist comprises a human VISTA fusion polypeptide, e.g., a human VISTA-Ig fusion protein and/or a human VSIG3 fusion polypeptide, e.g., a human VSIG3-Ig fusion protein.

The method of any of the foregoing claims wherein the VISTA agonist comprises a human Fc region, e.g., a human IgG1, IgG2, IgG3 or IgG4 Fc region.

In any of the foregoing exemplary embodiments, the VISTA agonist comprises a human IgG2 Fc region.

In any of the foregoing exemplary embodiments, the VISTA agonist comprises a human Fc region, e.g., a human IgG1, IgG2, IgG3 or IgG4 Fc region, that has been mutated to alter (increase or decrease) at least one effector function, e.g., FcR binding, complement binding, glycosylation, or ADCC.

In any of the foregoing exemplary embodiments, the VISTA agonist comprises a human IgG2 Fc region which binds to all or at least one Fc receptor bound by an endogenous human IgG2 Fc region.

In any of the foregoing exemplary embodiments, the levels of at least one cytokine or anti-inflammatory molecule or proinflammatory molecule, e.g., CXCL10, CXCR2, IL-6, CRP, gamma interferon, IL-1, TNF, IFN-γ, IL-2, IL-17, CCL5/Rantes, CCL3/MIP-1alpha, and CXCL11/I-TAC in the patient are detected prior to treatment.

In any of the foregoing exemplary embodiments, the levels of said cytokine or proinflammatory molecule in the patient are detected and confirmed to be aberrant prior to treatment.

In any of the foregoing exemplary embodiments, the levels of VISTA in the patient are detected prior to and/or after treatment.

In any of the foregoing exemplary embodiments, the levels of at least one of CXCL10, CXCR2, IL-6, CRP, IFN-γ, IL-2, IL-17, CCL5/Rantes, CCL3/MIP-1alpha, and CXCL11/I-TAC in the patient are detected prior to and/or after treatment.

In any of the foregoing exemplary embodiments, the levels of IL-6 and/or CRP and/or any of IFN-γ, IL-2, IL-17, CCL5/Rantes, CCL3/MIP-1alpha, and CXCL11/I-TAC in the patient are detected and confirmed to be elevated prior to treatment.

In any of the foregoing exemplary embodiments, the VISTA agonist is administered at a dose ranging from 0.01-5000 mg, 1-1000 mg, 1-500 mg, 5 mg-50 mg or about 1-25 mg.

In any of the foregoing exemplary embodiments, the VISTA agonist is administered every 2 or 3 days, biweekly, weekly, every 2 or 3 weeks, or every 4 weeks intravenously or via subcutaneous injection.

In any of the foregoing exemplary embodiments, the patient receives another anti-inflammatory treatment, optionally one or more of corticosteroids; inhaled nitric oxide (NO); extracorporeal membrane oxygenation (venovenous or venoarterial) or another immunosuppressive agent, optionally thymoglobulin, basiliximab, mycophenolate mofetil, tacrolimus, an anti-CD20 mAb such as rituximab, or a corticosteroid.

In any of the foregoing exemplary embodiments, the patient is additionally treated with another biologic, e.g. another antibody that targets a checkpoint protein such as PD-1, PD-L1, PD-L2, CTLA-4 or an IL-6 antagonist.

In any of the foregoing exemplary embodiments, the anti-VISTA antibody or antibody fragment contains an Fc region that has been modified to alter effector function, half-life, proteolysis, and/or glycosylation.

In any of the foregoing exemplary embodiments, the anti-VISTA antibody is selected from a humanized, single chain, or chimeric antibody and the antibody fragment is selected from a Fab, Fab′, F(ab′)2, Fv, or scFv.

In any of the foregoing exemplary embodiments, the patient aberrantly expresses one or more biomarkers correlated with the onset or increased risk of stroke or myocardial infarction, wherein said biomarkers optionally include low-density lipoprotein-cholesterol, hemoglobin A1c (HgA1c), C-reactive protein, lipoprotein-associated phospholipase A2, urinary albumin excretion, natriuretic peptides, glial fibrillary acidic protein, S100b, neuron-specific enolase, myelin basic protein, interleukin-6, matrix metalloproteinase (MMP)-9, D-dimer, and fibrinogen.

### BRIEF DESCRIPTION OF THE FIGURES

FIG. 1 shows the study design for an animal study (effected in in Male C57BL/6 mice (6-8 weeks old)) evaluating the efficacy of anti-VISTA antibodies to treat or prevent kidney injury due to ischemia followed by reperfusion. In the study male C57BL/6 mice (6-8 weeks old) were assigned to two groups (n=6 per group) and pretreated with anti-VISTA (250 μg/treatment, i.p.) or a control antibody (250 μg/treatment, i.p.) and from which plasma samples were collected 72 hours after IR injury to examine the severity of renal dysfunction by measurements of plasma creatinine and blood urea nitrogen (BUN).

FIG. 2 shows the results of the animal study depicted schematically in FIG. 1. The results therein show that the levels of plasma creatinine and BUN were significantly decreased in anti-VISTA-treated mice subjected to 30 minute renal IR compared to control-treated mice (FIG. 2A-B, n=3).

FIG. 3A-D shows the results of morphological assessments of the treated animals. In these assessments hematoxylin and eosin (H&E) staining was performed by an experienced renal pathologist, who was unaware of the treatment that each animal had received. As shown in FIG. 3A, severe tubular necrosis (FIG. 3A, red arrow) was observed in control-treated mice, whereas significantly less tubular epithelial cell necrosis (FIG. 3B, red arrow) and no whole tubular necrosis was observed in the anti-VISTA-treated mice. Moreover, as shown in FIG. 3C the mice who were treated with anti-VISTA showed a decrease in the renal tubular injury score (FIG. 3C) compared with control-treated mice (FIG. 3C). Also, as shown in FIG. 3D kidney inflammation after renal IR was assessed by the detection of inflammatory cell infiltration using H&E staining 72 hours after IR. The inflammatory infiltration score in the kidney was based on an established grading scale of inflammatory cell infiltration (0-3, inflammatory infiltration score (FIG. 3D). As shown in FIG. 3D the mice who were treated with anti-VISTA showed a decrease in the renal interstitial inflammation score.

FIG. 4A-C shows that in the same kidney injury model, many MPO-positive stained cells (FIG. 4A, red arrow) were observed in control-treated mice, especially in the glomeruli. By contrast, few MPO-positive stained cells (FIG. 4B, red arrow) were observed in anti-VISTA-treated mice, and no MPO-positive stained cells were observed in the glomeruli. Results were quantified and expressed as the number of positive MPO-positive cells/0.05 mm² (FIG. 4C).

FIG. 5 depicts schematically the likely sequence of events involved in ischemia and reperfusion injury.

FIG. 6 contains the CDR and variable heavy and light chain polypeptide sequences of exemplary agonistic anti-human VISTA antibodies.

FIG. 7 schematically describes a proposed clinical trial relating to the use of a VISTA agonist to treat DGF in kidney transplant.

FIG. 8 schematically describes a proposed clinical trial relating to the use of a VISTA agonist to treat cardiac surgery-associated acute kidney injury, myocardial infarction and ischemic stroke.

FIG. 9 schematically describes a proposed clinical trial relating to the use of a VISTA agonist to treat myocardial infarction.

FIG. 10 schematically describes a proposed clinical trial relating to the use of a VISTA agonist to treat ischemic stroke.

### SUMMARY

This invention is directed to treating inflammatory conditions mediated by overexpression of innate derived cytokines and chemokines optionally one or more of IL-1α, IL-6, TNF-α, IFN-γ, and granulocyte-monocyte colony stimulating factor (GM-CSF), IP-10 and others, many of which are driven by the IFNI response.

This invention specifically relates to the use of VISTA agonists for the treatment and prevention of ischemic reperfusion injury (IRI) and conditions associated therewith including myocardial infarction, cardiac surgery, stroke, solid organ transplant and post-surgery acute kidney injury.

### DETAILED DESCRIPTION

Prior to disclosing the invention in more detail the following definitions are provided.

### Definitions

As used herein “VISTA” or “V-domain Ig suppressor of T cell activation (VISTA)” refers to a type I transmembrane protein that functions as an immune checkpoint and is encoded by the C10orf54 gene. VISTA is produced at high levels in tumor-infiltrating lymphocytes, such as myeloid-derived suppressor cells and regulatory T cells, and its blockade with an antibody results in delayed tumor growth in mouse models of melanoma and squamous cell carcinoma. Monocytes from HIV-infected patients produce higher levels of VISTA compared to uninfected individuals. Increased VISTA levels correlated with an increase in immune activation and a decrease in CD4-positive T cells. VISTA further includes human, non-human primate, murine and other mammalian forms of VISTA.

As used herein, a “VISTA Agonist” refers to any molecule which specifically and directly agonizes (promotes) the expression of VISTA and/or which promotes or increases at least one functional activity of VISTA, e.g., its suppressive effects on T cell immunity (CD8⁺ T cell or CD4⁺ T cell immunity) and its suppressive effect on Foxp3 expression and/or its suppressive or promoting effect on the expression of cytokines, anti-inflammatory and proinflammatory molecules, particularly VISTA's modulatory (decrease or increase) effect on the expression of specific cytokines, activation markers and other immune molecules, e.g., those where expression is by or regulated by T cells. VISTA elicits effects on the expression and activity of specific immune molecules including specific cytokines such as IFN-γ, IL-2, IL-17, CCL5/Rantes, CCL3/MIP-1alpha, and CXCL11/I-TAC. VISTA agonists herein specifically include VISTA fusion proteins, agonist anti-VISTA antibodies and agonist antibody fragments which directly promote VISTA's effects on one or more of these molecules. Also, as VSIG3 reportedly is a ligand for VISTA (see Jinghua Wang, Guoping Wu, Brian Manick, Vida Hernandez, Mark Renelt, Ming Bi, Jun Li and Vassilios Kalabokis, J Immunol May 1, 2017, 198 (1 Supplement) 154.1); which when bound to VISTA promotes its activity, VISTA agonists herein further include compounds (VSIG3 fusion proteins, anti-VSIG3 antibodies and antibody fragments) which directly promote VISTA's effects on one or more of these molecules. Herein VSIG-3 also referred to as IGSF11 includes human, non-human primate, murine and other mammalian forms of VSIG-3. Also VISTA agonists include other moieties that provide for increased VISTA expression or amounts in a subject, e.g., cells engineered to express VISTA, e.g., under controllable conditions or compounds which promote the expression of VISTA. Further, VISTA agonists include anti-PSLG1 antibodies and antibody fragments and PSGL1 fusion proteins and small molecules which agonize the VISTA/PSGL1 binding interaction. In this regard PSGL1 has been reported to be a binding partner of VISTA (see WO 2018/169993 filed by Bristol Myers and Robert J. Johnston et al., “VISTA is an acidic pH-selective ligand for PSGL-1”, Nature (2019) 574: 565-570.

As used herein, “Cytokine Storm” or “Hypertyrosinemia” or Cytokine Release Syndrome” or “CRS” refers to a severe immune reaction in which the body releases too many cytokines into the blood too quickly. Cytokines play an important role in normal immune responses, but having a large amount of them released in the body all at once can be harmful. A cytokine storm can occur as a result of an infection, autoimmune condition, or other disease. It may also occur after treatment with some types of immunotherapy. Signs and symptoms include high fever, inflammation (redness and swelling), and severe fatigue and nausea. Sometimes, a cytokine storm may be severe or life threatening and lead to multiple organ failure. The pathogenesis is complex but includes loss of regulatory control of proinflammatory cytokine production, both at local and systemic levels. The disease progresses rapidly, and the mortality is high. For example COVID-19 infection has been closely associated with dysregulated and excessive cytokine release or “cytokine storm”.

As used herein, “improved,” “improvement,” and other grammatical variants, includes any beneficial change resulting from a treatment. A beneficial change is any way in which a patient's condition is better than it would have been in the absence of the treatment. “Improved” includes prevention of an undesired condition, slowing the rate at which a condition worsens, delaying the development of an undesired condition, and increasing the rate at which a desired condition is reached. For example, improvement in an ARDS patient encompasses any decrease in inflammatory cytokines as any increase in the amount or rate at which inflammatory cytokines are prevented, removed or reduced. For another example, improvement in a ARDS patient or patient at risk of ARDS encompasses any prevention, decrease, delay or slowing in the rate of the condition and cytokine mediated damage or loss of function, e.g., to lung function.

The term “antibody” or “Ab,” or “immunoglobulin” is used herein in the broadest sense and encompasses various antibody structures which specifically binds with an antigen, including but not limited to monoclonal antibodies, polyclonal antibodies, multispecific antibodies (e.g., bispecific antibodies), and/or antibody fragments (also referred to as “antigen-binding antibody fragments”). Typically, a full-size Ab (also referred to as an intact Ab) comprises two pairs of chains, each pair comprising a heavy chain (HC) and a light chain (LC). A HC typically comprises a variable region and a constant region. A LC also typically comprises a variable region and constant region. The variable region of a heavy chain (VH) typically comprises three complementarity-determining regions (CDRs), which are referred to herein as CDR 1, CDR 2, and CDR 3 (or referred to as CDR-H1, CDR-H2, CDR-H3, respectively). The constant region of a HC typically comprises a fragment crystallizable region (Fc region), which dictates the isotype of the Ab, the type of Fc receptor the Ab binds to, and therefore the effector function of the Ab. Any isotype, such as IgG1, IgG2a, IgG2b, IgG3, IgG4, IgM, IgD, IgE, IgGA1, or IgGA2, may be used. Fc receptor types include, but are not limited to, FcaR (such as FcaRI), Fca/mR, FceR (such as FceRI, FceRII), FcgR (such as FcgRI, FcgRIIA, FcgRIIB1, FcgRIIB2, FcgRIIIA, FcgRIIIB), and FcRn and their associated downstream effects are well known in the art. The variable region of a light chain (VL) also typically comprises CDRs, which are CDR 1, CDR 2, and CDR 3 (or referred to as CDR-L1, CDR-L2, CDR-L3, respectively). In some embodiments, the antigen is ACVR1C (also referred to as ALK7). Antibodies can be intact immunoglobulins derived from natural sources or from recombinant sources. A portion of an antibody that comprises a structure that enables specific binding to an antigen is referred to “antigen-binding fragment,” “AB domain,” “antigen-binding region,” or “AB region” of the Ab.

Certain amino acid modifications in the Fc region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9(1): 63-73; Dall'Acqua W. F. et al., J Biol Chem. 2006 Aug. 18; 281(33):23514-24. Epub 2006 Jun. 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289(6): 3571-3590).

An IgG1-type Fc optionally may comprise one or more amino acid substitutions. Such substitutions may include, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y, M428L, N434S, L328F, M252Y, S254T, T256E, and/or any combination thereof (the residue numbering is according to the EU index as in Kabat) (Dall'Acqua W. F. et al., J Biol Chem. 2006 Aug. 18; 281(33):23514-24. Epub 2006 Jun. 21; Wang X. et al., Protein Cell. 2018 January; 9(1): 63-73), or for example, N434A, Q438R, S440E, L432D, N434L, and/or any combination thereof (the residue numbering according to EU numbering). The Fc region may further comprise one or more additional amino acid substitutions. Such substitutions may include but are not limited to A330L, L234F, L235E, P3318, and/or any combination thereof (the residue numbering is according to the EU index as in Kabat). Specific exemplary substitution combinations for an IgG1-type Fc include, but not limited to: M252Y, S254T, and T256E (“YTE” variant); M428L and N434A (“LA” variant), M428L and N434S (“LS” variant); M428L, N434A, Q438R, and S440E (“LA-RE” variant); L432D and N434L (“DEL” variant); and L234A, L235A, L432D, and N434L (“LALA-DEL” variant) (the residue numbering is according to the EU index as in Kabat).

When the Ab is an IgG2, the Fc region optionally may comprise one or more amino acid substitutions. Such substitutions may include but are not limited to P238S, V234A, G237A, H268A, H268Q, H268E, V309L, N297A, N297Q, A330S, P331S, C232S, C233S, M252Y, S254T, T256E, and/or any combination thereof (the residue numbering is according to the EU index as in Kabat). The Fc region optionally may further comprise one or more additional amino acid substitutions. Such substitutions may include but are not limited to M252Y, S254T, T256E, and/or any combination thereof (the residue numbering is according to the EU index as in Kabat). Preferably when the Ab is an IgG2, the Fc region will comprise native (unmodified) FcR binding.

An IgG3-type Fc region optionally may comprise one or more amino acid substitutions. Such substitutions may include but are not limited to E235Y (the residue numbering is according to the EU index as in Kabat).

An IgG4-type Fc region optionally may comprise one or more amino acid substitutions. Such substitutions may include but are not limited to, E233P, F234V, L235A, G237A, E318A, S228P, L236E, S241P, L248E, T394D, M252Y, S254T, T256E, N297A, N297Q, and/or any combination thereof (the residue numbering is according to the EU index as in Kabat). The substitution may be, for example, S228P (the residue numbering is according to the EU index as in Kabat).

In some cases, the glycan of the human-like Fc region may be engineered to modify the effector function (for example, see Li T. et al., Proc Natl Acad Sci USA. 2017 Mar. 28; 114(13):3485-3490. doi: 10.1073/pnas.1702173114. Epub 2017 Mar. 13).

The term “antibody fragment” or “Ab fragment” as used herein refers to any portion or fragment of an Ab, including intact or full-length Abs that may be of any class or sub-class, including IgG and sub-classes thereof, IgM, IgE, IgA, and IgD. The term encompasses molecules constructed using one or more potions or fragments of one or more Abs. An Ab fragment can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term also encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In a specific embodiment, the antibody fragment is a scFv. Unless otherwise stated, the term “Ab fragment” should be understood to encompass functional antibody fragments thereof. A portion of an Ab fragment that comprises a structure that enables specific binding to an antigen is referred to as “antigen-binding Ab fragment,” “AB domain,” “antigen-binding region,” or “antigen-binding region” of the Ab fragment.

The term “humanization” of an Ab refers to modification of an Ab of a non-human origin to increase the sequence similarity to an Ab naturally produced in humans. The term “humanized antibody” as used herein refers to Abs generated via humanization of an Ab. Generally, a humanized or engineered antibody has one or more amino acid residues from a source which is non-human, e.g., but not limited to mouse, rat, rabbit, non-human primate or other mammal. These human amino acid residues are often referred to as “import” residues, which are typically taken from an “import” variable, constant or other domain of a known human sequence. Known human Ig sequences are disclosed, e.g., www.ncbi.nlm.nih.gov/entrez/query.fcgi; www.atcc.org/phage/hdb.html, each entirely incorporated herein by reference. Such imported sequences can be used to reduce immunogenicity or reduce, enhance or modify binding, affinity, avidity, specificity, half-life, or any other suitable characteristic, as known in the art. Generally part or all of the non-human or human CDR sequences are maintained while part or all of the non-human sequences of the framework and/or constant regions are replaced with human or other amino acids. Antibodies can also optionally be humanized with retention of high affinity for the antigen and other favorable biological properties using three-dimensional immunoglobulin models that are known to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, for example, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824514, 5,817483, 5,814476, 5,763,192, 5,766,886, 5,714,352, 6,187,287, 6,204,023, 6,180,370, 5,693,762, 5,585,089, 5,225,539; 4,816,567, each entirely incorporated herein by reference, included references cited therein.

An “isolated” biological component (such as an isolated protein, nucleic acid, vector, or cell) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

The term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The term “ischemia” refers to a state or condition in which the blood flow (and thus oxygen) is restricted or reduced in a part of the body. For example cardiac ischemia is the name for decreased blood flow and oxygen to the heart muscle. Ischemia or ischaemia may comprise a restriction in blood supply to any tissues, muscle group, or organ of the body, causing a shortage of oxygen that is needed for cellular metabolism (to keep tissue alive). Ischemia is generally caused by problems with blood vessels, with resultant damage to or dysfunction of tissue, i.e., hypoxia and microvascular dysfunction. It also includes local hypoxia in a given part of a body sometimes resulting from constriction (such as vasoconstriction, thrombosis or embolism). Ischemia comprises not only insufficiency of oxygen, but also reduced availability of nutrients and inadequate removal of metabolic wastes. Ischemia can be partial (poor perfusion) or total blockage. The inadequate delivery of oxygenated blood to the organs must be resolved either by treating the cause of the inadequate delivery or reducing the oxygen demand of the system that needs it. For example, patients with myocardial ischemia have a decreased blood flow to the heart and are prescribed with medications that reduce chronotrophy and ionotrophy to meet the new level of blood delivery supplied by the stenosed so that it is adequate.

The term “pharmaceutically acceptable excipient,” “pharmaceutical excipient,” “excipient,” “pharmaceutically acceptable carrier,” “pharmaceutical carrier,” or “carrier” as used herein refers to compounds or materials conventionally used in pharmaceutical compositions during formulation and/or to permit storage. Excipients included in the formulations will have different purposes. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

The term “recombinant” means a polynucleotide, a protein, a cell, and so forth with semi-synthetic or synthetic origin which either does not occur in nature or is linked to another polynucleotide, a protein, a cell, and so forth in an arrangement not found in nature.

The term “reperfusion” refers to the action of restoring the flow of blood to an organ or tissue, typically after a heart attack or stroke.

The term “reperfusion injury” includes any injury resulting from restoring the flow of blood to an organ or tissue, typically after a heart attack or stroke. Examples include Hypoxic brain injury; multiple organ failure; acute kidney injury, Acute chest syndrome; pulmonary hypertension, priapism, acute kidney injury, Hypertension; diabetes

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. The linker may comprise portions of the framework sequences. In scFvs, the heavy chain variable domain (HC V, HCV, or VH) may be placed upstream of the light chain variable domain (LC V, LCV, or VL), and the two domains may optionally be linked via a linker (for example, the G4S X3 linker). Alternatively, the heavy chain variable domain may be placed downstream of the light chain variable domain, and the two domains may optionally be linked via a linker (for example, the G4S X3 linker).

The term “subject” as used herein may be any living organisms, preferably a mammal. In some embodiments, the subject is a primate such as a human. In some embodiments, the primate is a monkey or an ape. The subject can be male or female and can be any suitable age, including infant, juvenile, adolescent, adult, and geriatric subjects. In some examples, the patient or subject is a validated animal model for disease and/or for assessing toxic outcomes. The subject may also be referred to as “patient” in the art. The subject may have a disease or may be healthy.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. In other embodiments “treat”, “treatment,” or “treating” may result in the inhibition of the progression of a disease, either physically by, e.g., stabilization of a discernible symptom, physiologically by, e.g., stabilization of a physical parameter, or both. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

### DETAILED DESCRIPTION

This invention is based in part on an earlier discovery by Applicant that VISTA negatively regulates innate inflammation through the transcriptional and epigenetic re-programming of immune cells, particularly macrophages. Applicant demonstrated [results not shown herein] that VISTA agonists functionally and transcriptionally re-program macrophages by negatively regulating macrophage responses to proinflammatory stimuli. Particularly, anti-VISTA alone induced mediators involved in both M2 polarization and LPS tolerance including IL-10, miR-221, IRG1, A20, and MerTK and suppressed mediators of M1 polarization (reduced IRF5 and IRF8 expression at both the transcriptional and protein levels). The VISTA-mediated reduction in these transcription factors (TFs) diminished the expression of inflammatory genes including IL-12 family members, IL-6 and TNFα. Furthermore, anti-VISTA upregulated key mediators of LPS tolerance resulting in the enhanced survival of mice from endotoxin shock. Based thereon, VISTA agonists may be of therapeutic relevance in specific inflammatory settings, e.g., those associated with cytokine storm or CRS or sepsis and/or acute or chronic respiratory associated syndrome and respiratory conditions.

The present invention specifically relates to treatment and prevention of other inflammatory conditions, particularly ischemic reperfusion injury (IRI) and other conditions associated with ischemic reperfusion injury based on the demonstration herein that the administration of an agonist anti-VISTA antibody, prior to IR injury, mitigates the effects of kidney injury and protects the kidney.

Thus, in one embodiment the present invention provides methods of preventing or treating an injury to an organ caused by ischemia, followed by reperfusion, comprising the administration of a therapeutically effective amount of anti-VISTA to a subject in need thereof. Such an injury can be caused by surgery, most usually surgery involving major organs, including but not limited to, the kidney, liver, heart, lung, intestine, and aorta and would include, but is not limited to, coronary bypass, major vascular repair, liver resection, and transplantation of the kidney, liver, and lung.

As discussed in the Background of the Invention, IR injury can have other causes including, but not limited to, cardiopulmonary bypass during surgery, stroke, liver ischemia, kidney ischemia, aortic occlusion, myocardial occlusion, cardiac arrest, shock and trauma. Therefore, the invention also relates to the treatment and prevention of ischemic reperfusion injury (IRI) associated with myocardial infarction, cardiac surgery, stroke, solid organ transplant, post-surgery acute kidney injury, cardiopulmonary bypass surgery, liver ischemia, kidney ischemia, aortic occlusion, myocardial occlusion, cardiac arrest, shock and trauma.

This invention relates in particular to treating and preventing injury caused by ischemia and reperfusion by the administration of an anti-VISTA antibody. Ischemia and reperfusion (IR) injury is caused by the return of blood supply to a tissue (reperfusion) after a period of lack of oxygen (ischemia). During ischemia, there is an absence of oxygen and nutrients normally supplied by the blood. This creates a condition where the return of circulation results in inflammation and oxidative damage, rather than the expected restoration of normal function.

Cell death from ischemia or hypoxia with subsequent reperfusion injury, is a major clinical problem affecting virtually every organ in the body. In fact, post-operative organ dysfunction due to IR injury is a severe threat affecting almost all patients undergoing major surgical procedures, including cardiac surgery, liver transplantation, liver resection, renal transplantation, lung transplantation, aortic surgery, and major vascular repair. Moreover, injury of one organ due to ischemia or hypoxia with reperfusion is frequently associated with remote organ injury affecting distant organs. For example, patients who develop kidney injury due to renal IR frequently develop liver, intestine, and lung dysfunction as well as sepsis leading to extraordinarily high mortality (25-80%). IR injury can also result in patients requiring dialysis.

Renal IR injury is a frequent cause of acute kidney injury (AKI). Ischemic AKI is a clinical problem for patients subjected to major surgical procedures not only involving the kidney, but the liver, heart or aorta as well, and may lead to multi-organ dysfunction and systemic inflammation with extremely high mortality.

Reperfusion of ischemic tissues is often associated with microvascular injury, particularly due to increased permeability of capillaries and arterioles that lead to an increase of diffusion and fluid filtration across the tissues. Activated endothelial cells produce more reactive oxygen species but less nitric oxide following reperfusion, and the imbalance results in a subsequent inflammatory response. The inflammatory response is partially responsible for the damage of reperfusion injury. White blood cells, carried to the area by the newly returning blood, release a host of inflammatory factors such as interleukins as well as free radicals in response to tissue damage. The restored blood flow reintroduces oxygen within cells that damages cellular proteins, DNA, and the plasma membrane. Damage to the cell's membrane may in turn cause the release of more free radicals. Such reactive species may also act indirectly in redox signaling to turn on apoptosis. White blood cells may also bind to the endothelium of small capillaries, obstructing them and leading to more ischemia. Another hypothesis would be that normally, tissues contain free radical scavengers to avoid damage by oxidizing species normally contained in the blood. Ischemic tissue would have decreased function of these scavengers because of cell injury. Once blood flow is reestablished, oxygen species contained in the blood will damage the ischemic tissue because the function of the scavengers is decreased].

Reperfusion injury plays a major part in the biochemistry of hypoxic brain injury in stroke. Similar failure processes are involved in brain failure following reversal of cardiac arrest. Repeated bouts of ischemia and reperfusion injury also are thought to be a factor leading to the formation and failure to heal of chronic wounds such as pressure sores and diabetic foot ulcer. Continuous pressure limits blood supply and causes ischemia, and the inflammation occurs during reperfusion. As this process is repeated, it eventually damages tissue enough to cause a wound.

The main reason for the acute phase of ischemia-reperfusion injury is oxygen deprivation and, therefore, arrest of generation of ATP (cellular energy currency) by mitochondria oxidative phosphorylation. Tissue damage due to the general energy deficit during ischemia is followed by reperfusion (increase of oxygen level) when the injury is enhanced. Mitochondrial complex I is thought to be the most vulnerable enzyme to tissue ischemia/reperfusion but the mechanism of damage is different in different tissues. For example brain ischemia/reperfusion injury is mediated via complex I redox-dependent inactivation. It was found that lack of oxygen leads to conditions in which mitochondrial complex I lose its natural cofactor, flavin mononucleotide (FMN) and become inactive. When oxygen is present the enzyme catalyzes a physiological reaction of NADH oxidation by ubiquinone, supplying electrons downstream of the respiratory chain (complexes III and IV). Ischemia leads to dramatic increase of succinate level. In the presence of succinate mitochondria catalyze reverse electron transfer so that fraction of electrons from succinate is directed upstream to FMN of complex I. Reverse electron transfer results in a reduction of complex I FMN, increased generation of ROS, followed by a loss of the reduced cofactor (FMNH2) and impairment of mitochondria energy production. The FMN loss by complex I and I/R injury can be alleviated by the administration of FMN precursor, riboflavin.

In prolonged ischemia (60 minutes or more), hypoxanthine is formed as a breakdown product of ATP metabolism. The enzyme xanthine dehydrogenase acts in reverse, that is as a xanthine oxidase as a result of the higher availability of oxygen. This oxidation results in molecular oxygen being converted into highly reactive superoxide and hydroxyl radicals. Xanthine oxidase also produces uric acid, which may act as both a prooxidant and as a scavenger of reactive species such as peroxynitrite. Excessive nitric oxide produced during reperfusion reacts with superoxide to produce the potent reactive species peroxynitrite. Such radicals and reactive oxygen species attack cell membrane lipids, proteins, and glycosaminoglycans, causing further damage. They may also initiate specific biological processes by redox signaling. Also, reperfusion can cause hyperkalemia. Further, reperfusion injury is a primary concern in liver transplantation surgery.

As shown in the example infra, the inventors studied the effects of VISTA in the context of renal IR by using an anti-VISTA antibody to agonize VISTA signaling and potentially as a means of protecting against IR injury. We show herein that the administration of an agonistic anti-VISTA antibody, prior to IR injury, mitigates the effects of kidney injury and protects the kidney.

Thus, one embodiment of the present invention is a method of preventing or treating an injury to an organ caused by ischemia, followed by reperfusion, comprising the administration of a therapeutically effective amount of an anti-VISTA antibody to a subject in need thereof.

Particularly, such an injury can result from other causes such as surgery, most usually surgery involving major organs, including but not limited to, the kidney, liver, heart, lung, intestine, and aorta, coronary bypass, major vascular repair, liver resection, and transplantation of the kidney, liver, and lung. Also, IR injury can be mediated by other causes including, but not limited to, cardiopulmonary bypass during surgery, stroke, liver ischemia, kidney ischemia, aortic occlusion, myocardial occlusion, cardiac arrest, shock and trauma.

Therefore, the present invention addresses these needs by the treatment of subjects comprising or at risk of IRI by administering a therapeutically or prophylactically effective amount of a VISTA agonist, e.g., an agonist anti-VISTA agonist antibody effective to treat or prevent IRI and side effects thereof.

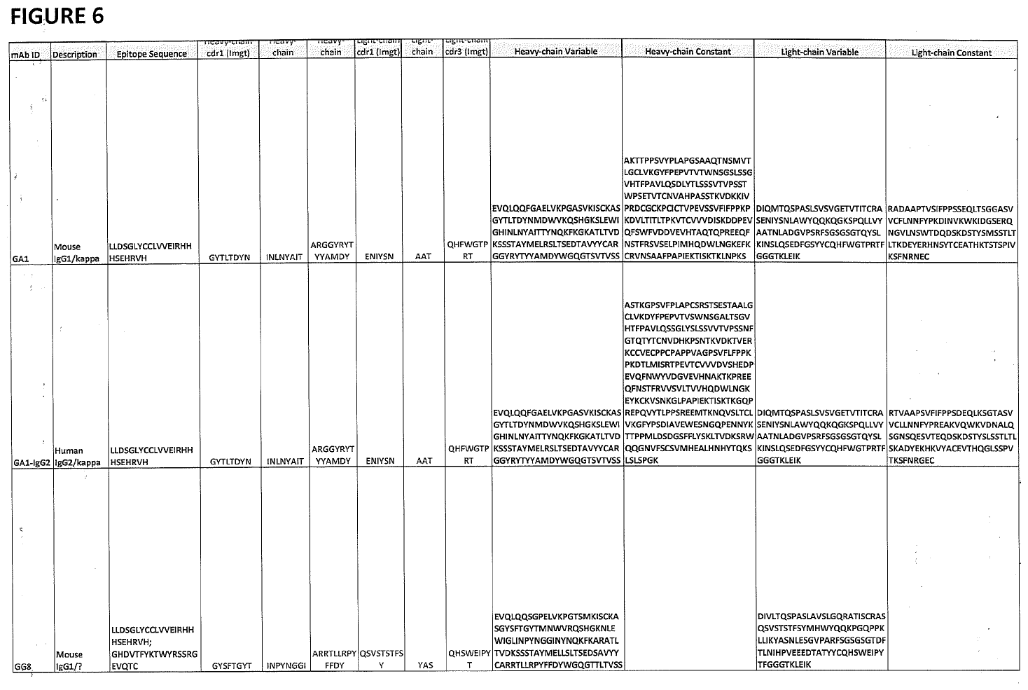

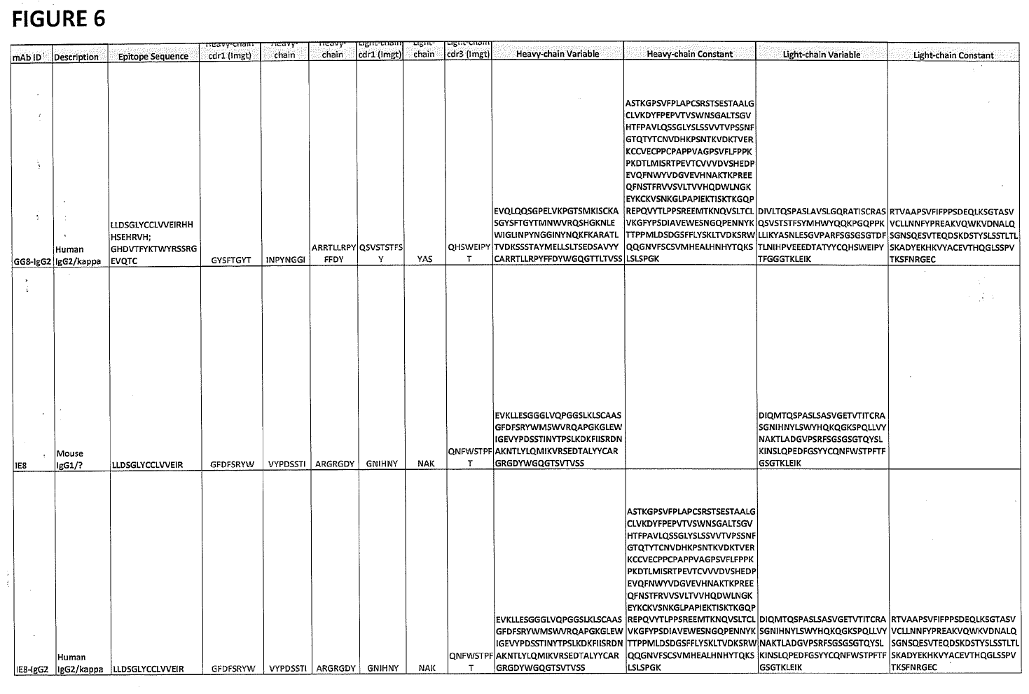

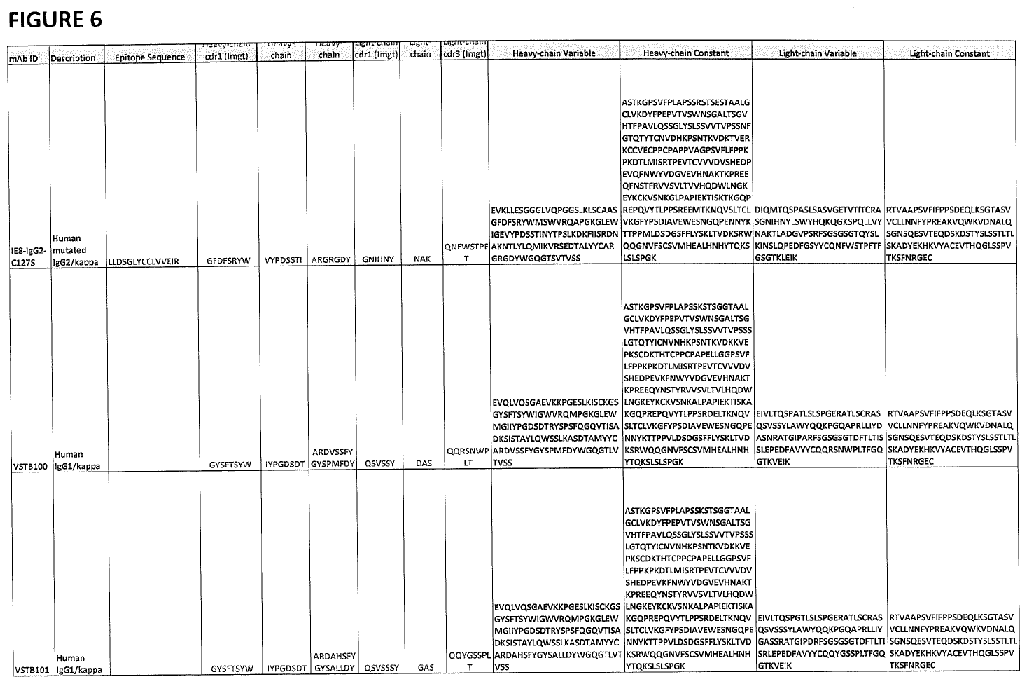

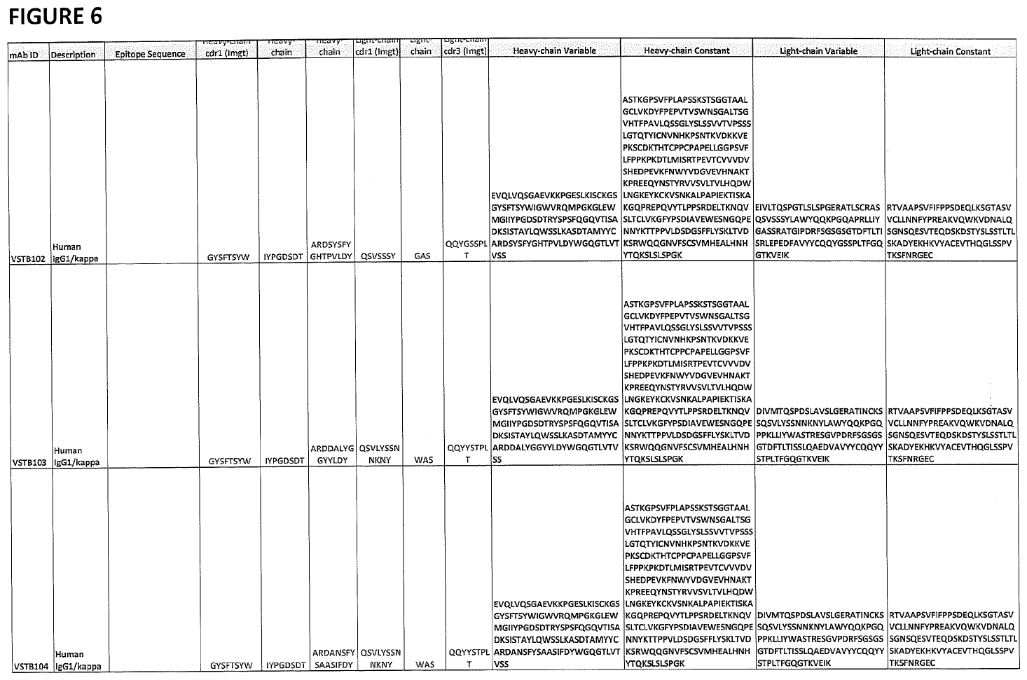

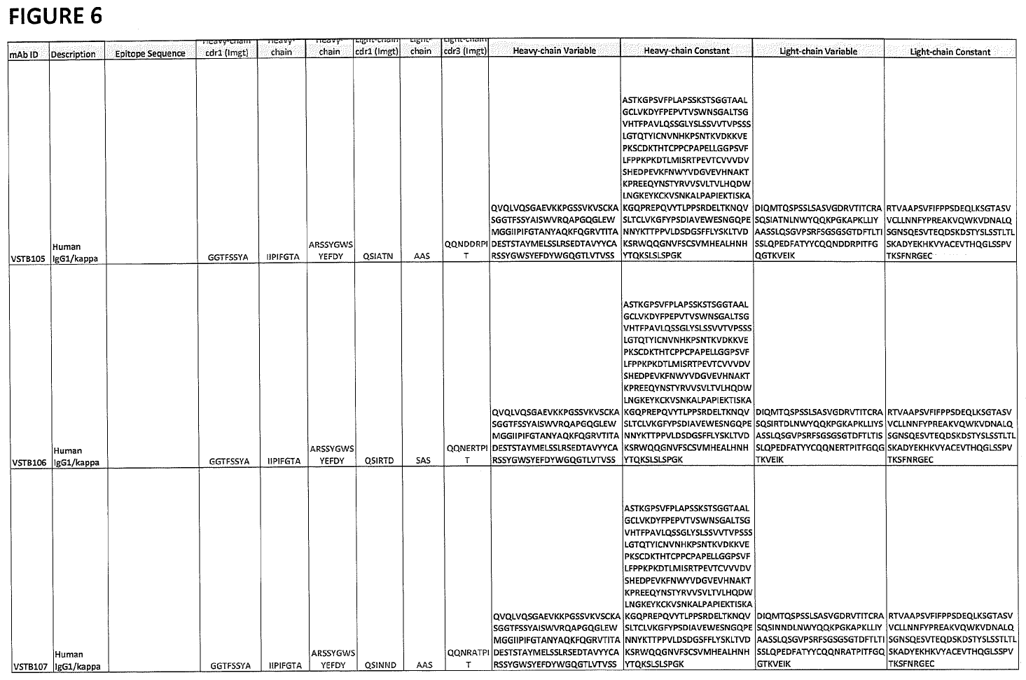

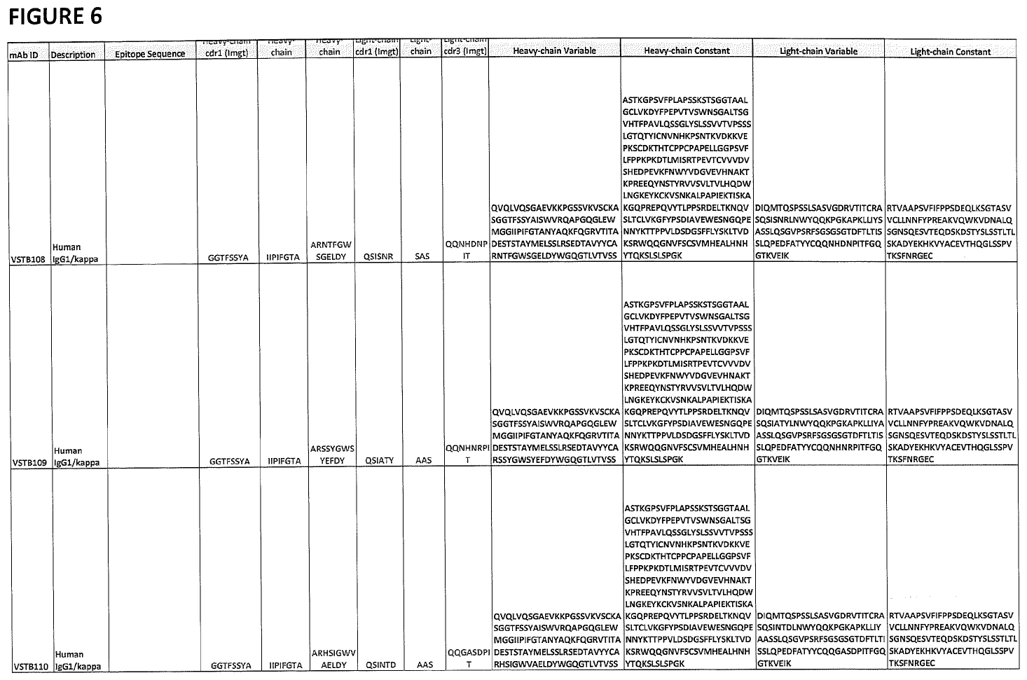

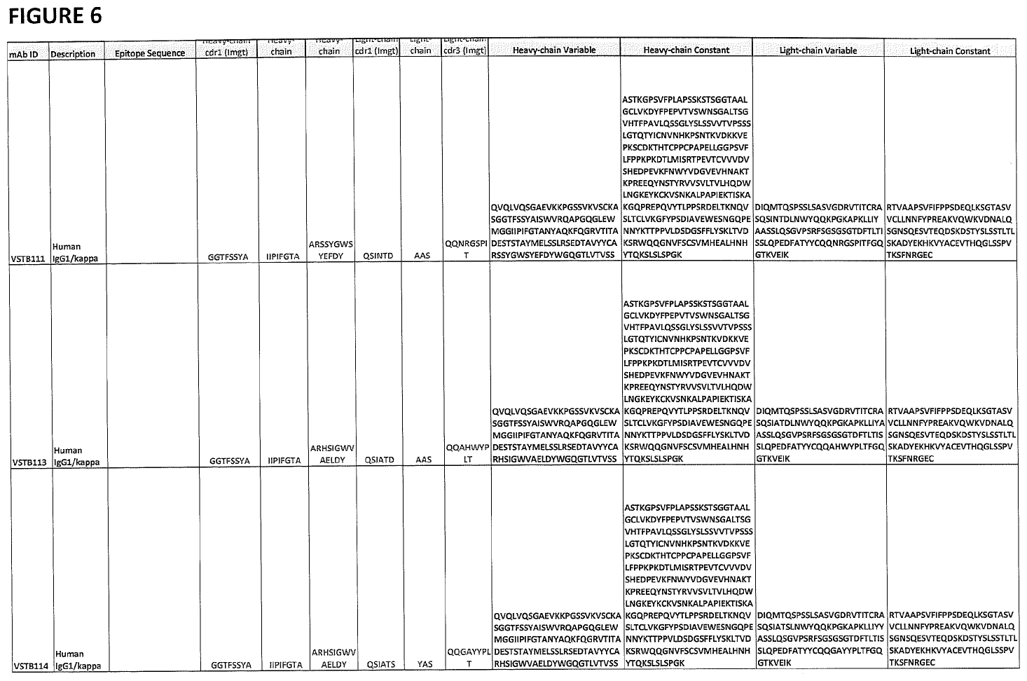

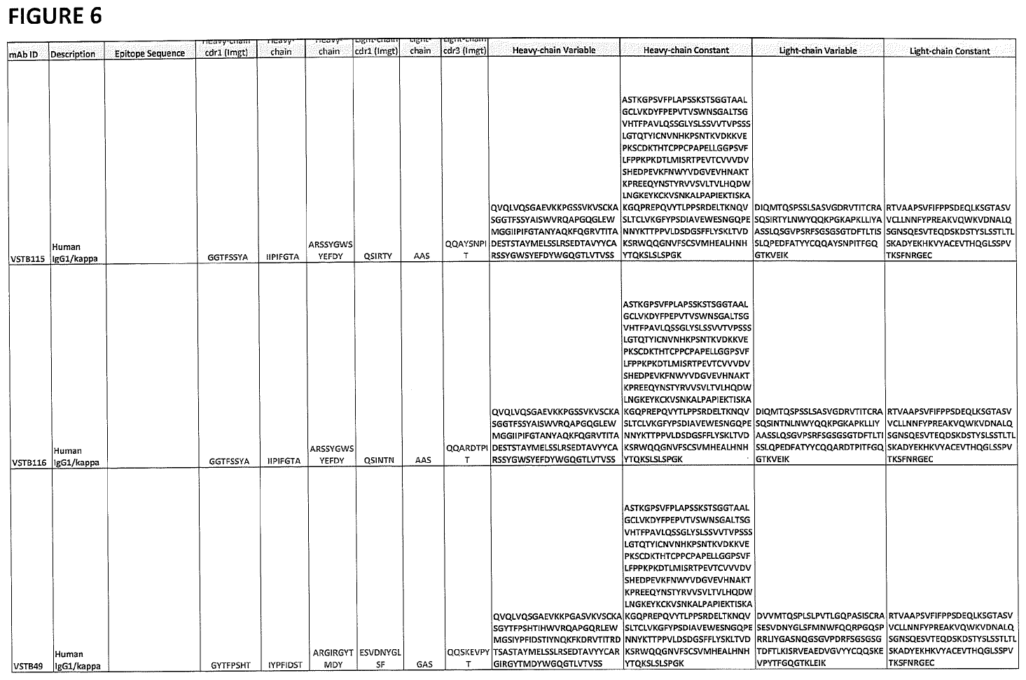

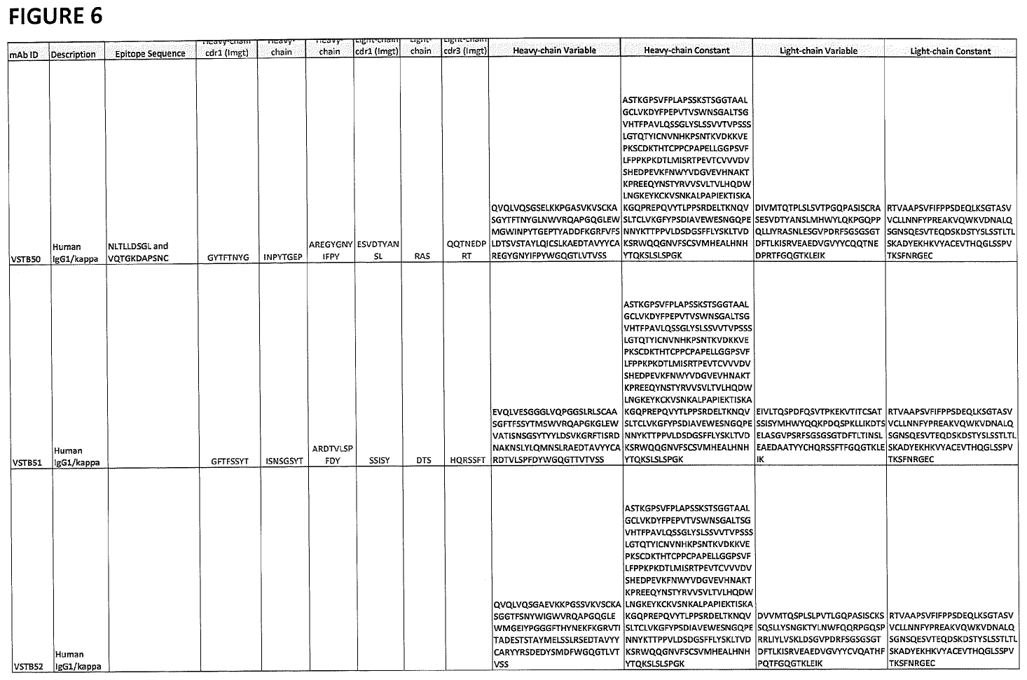

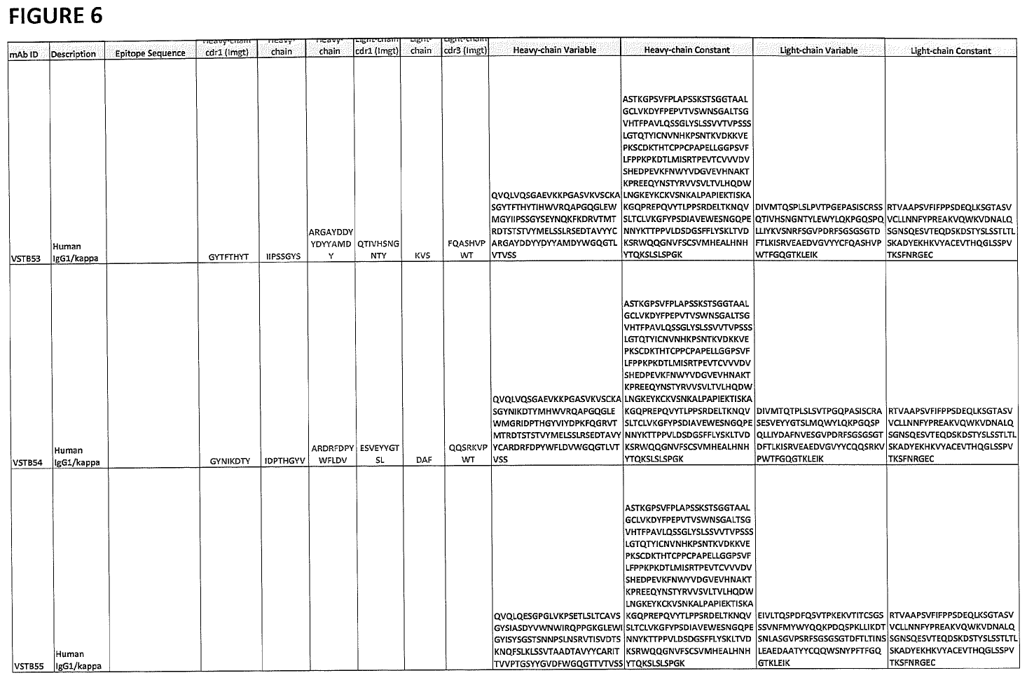

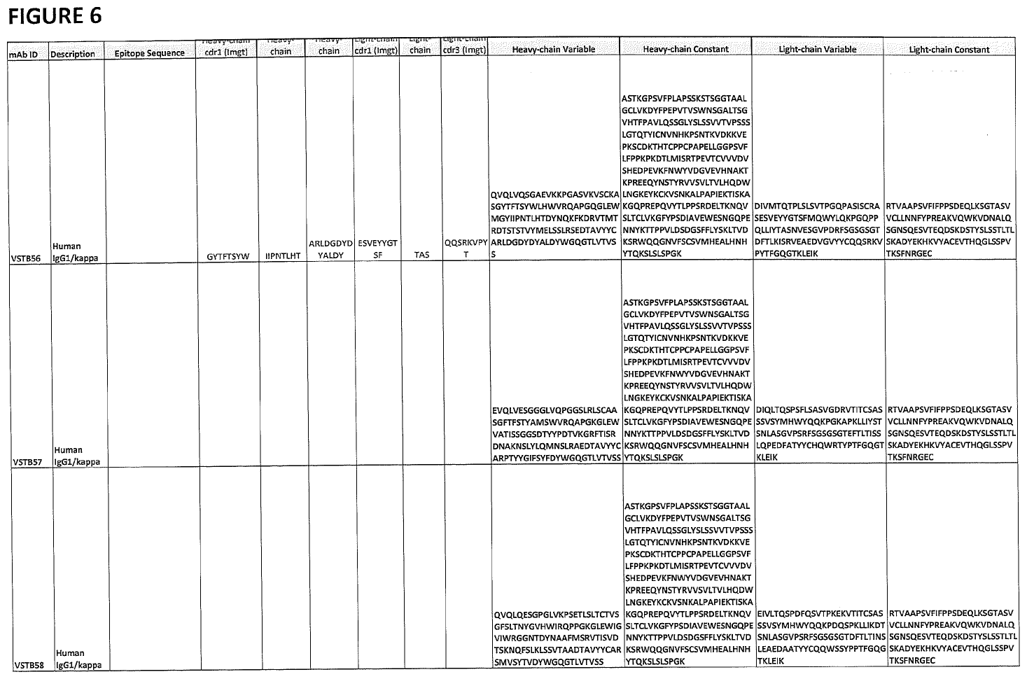

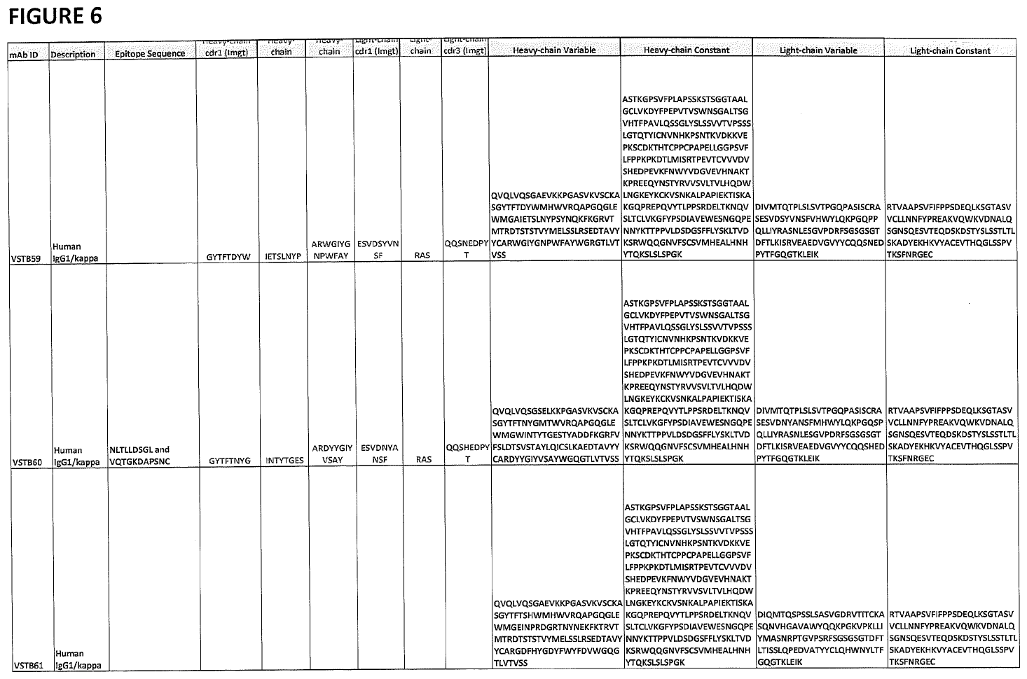

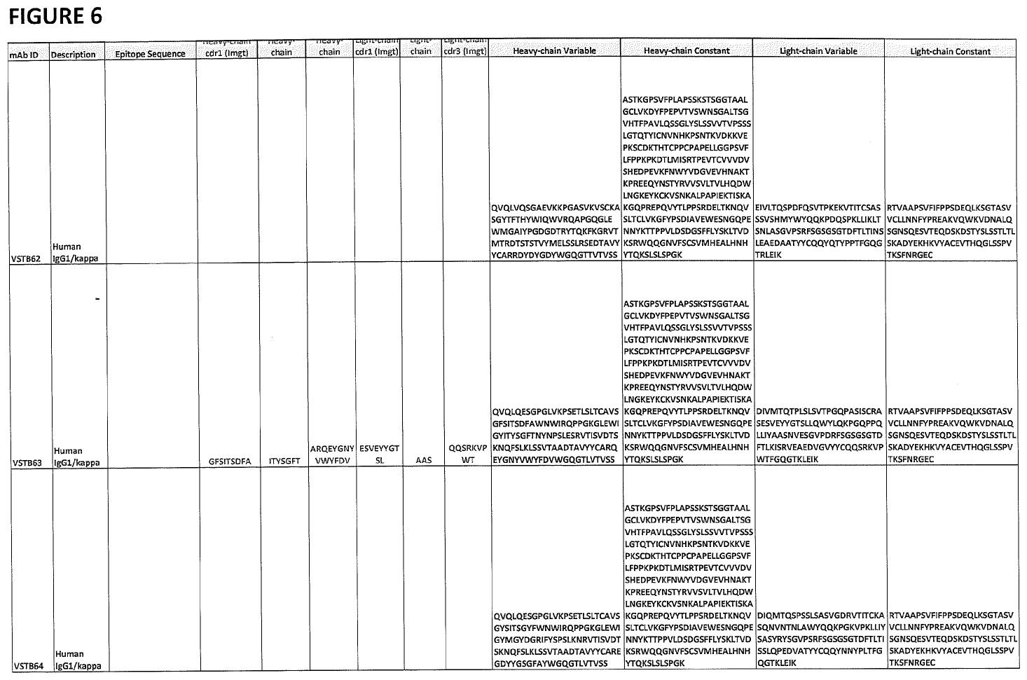

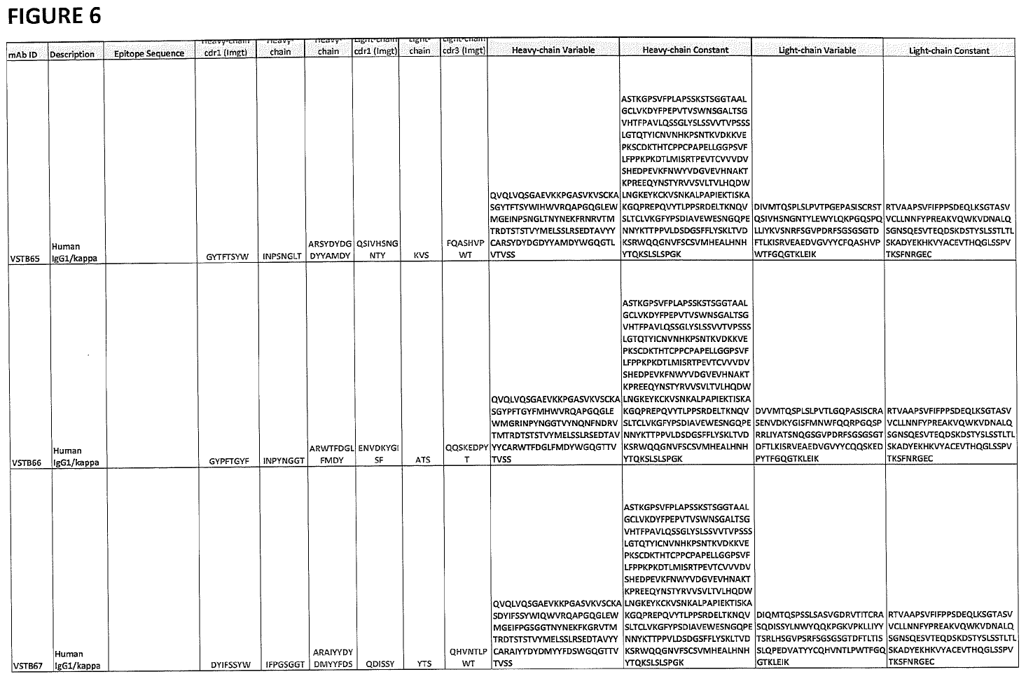

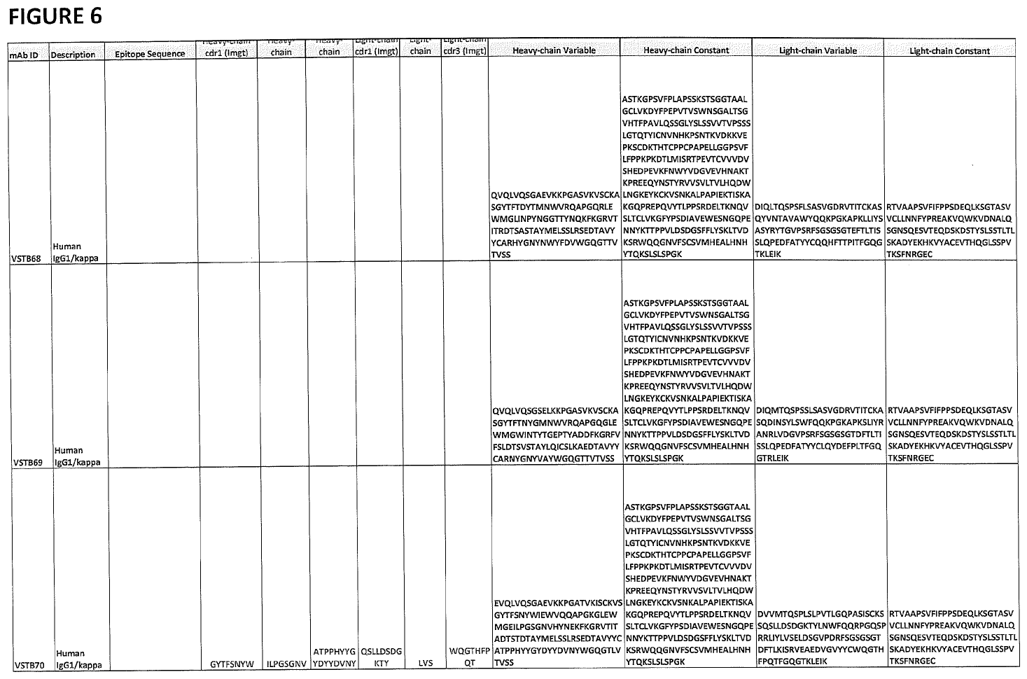

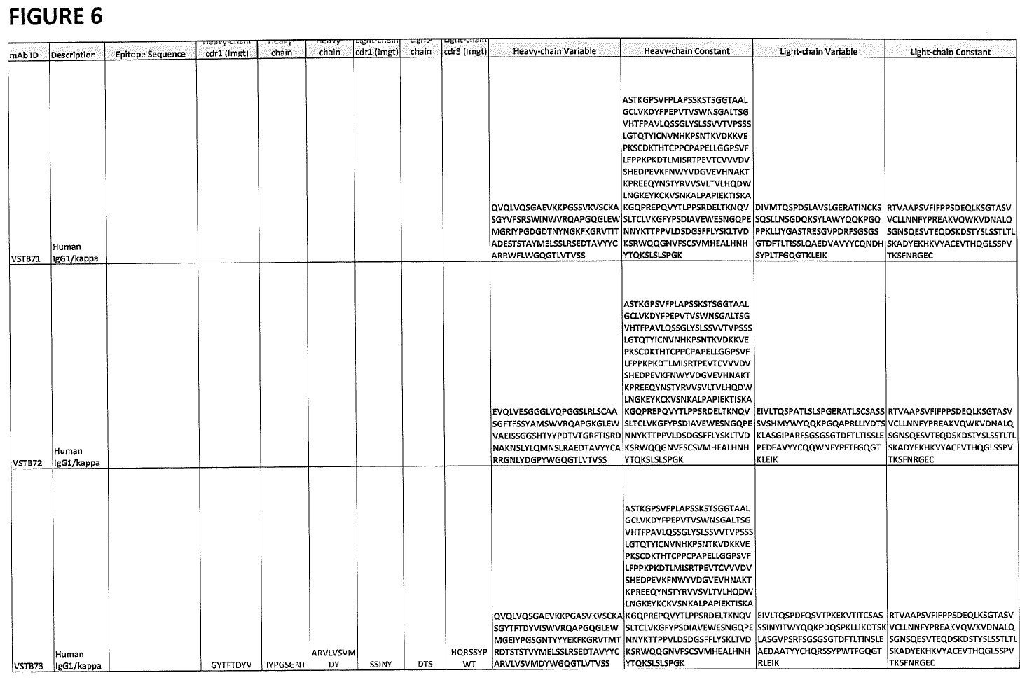

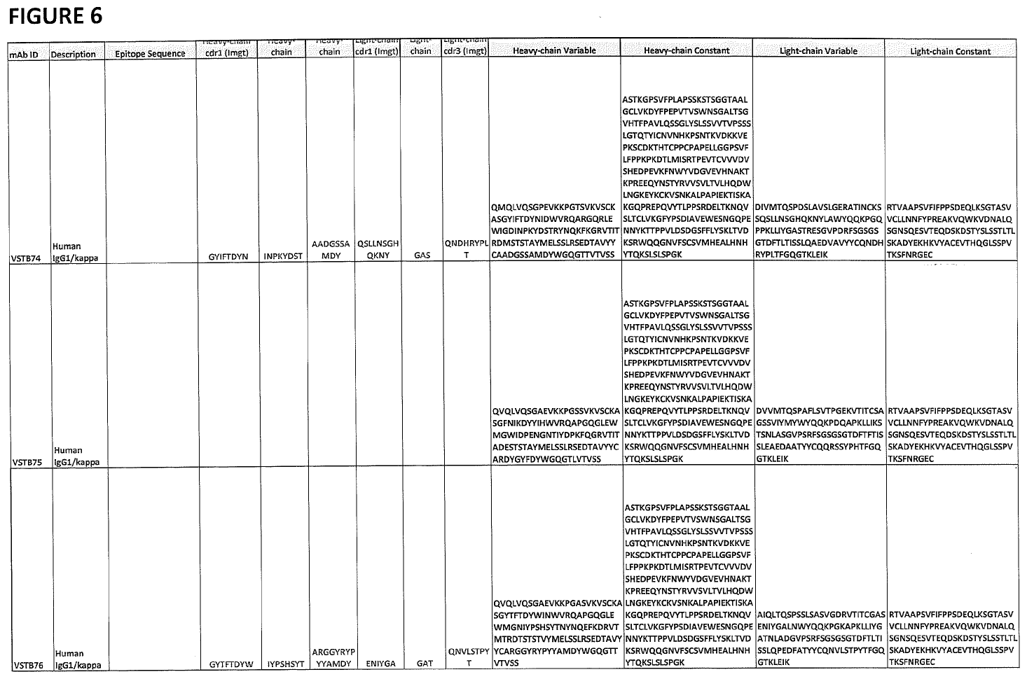

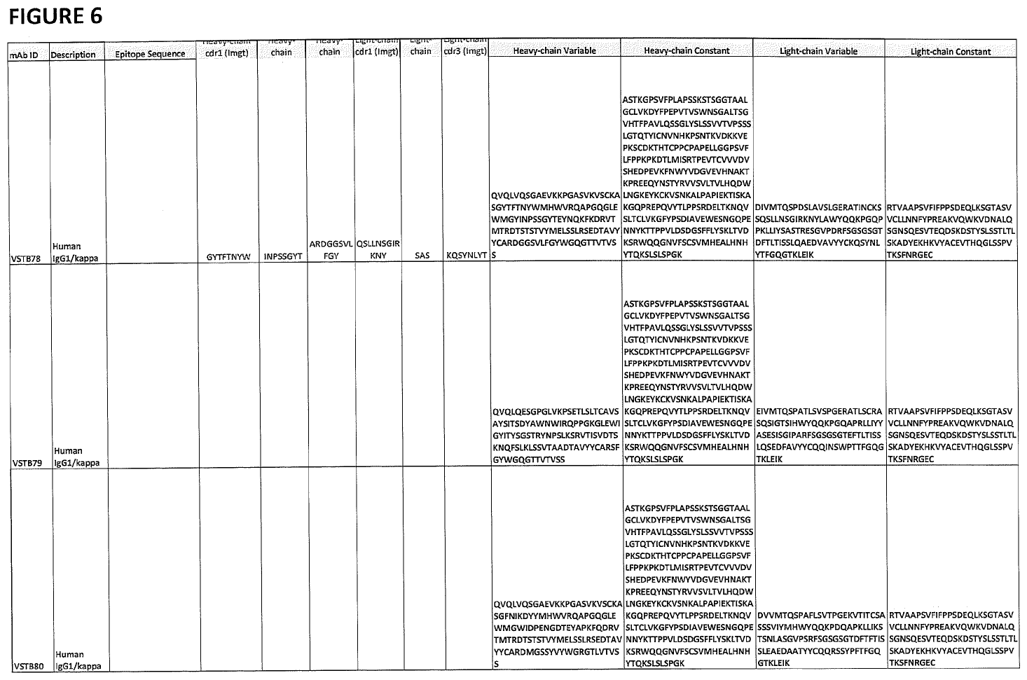

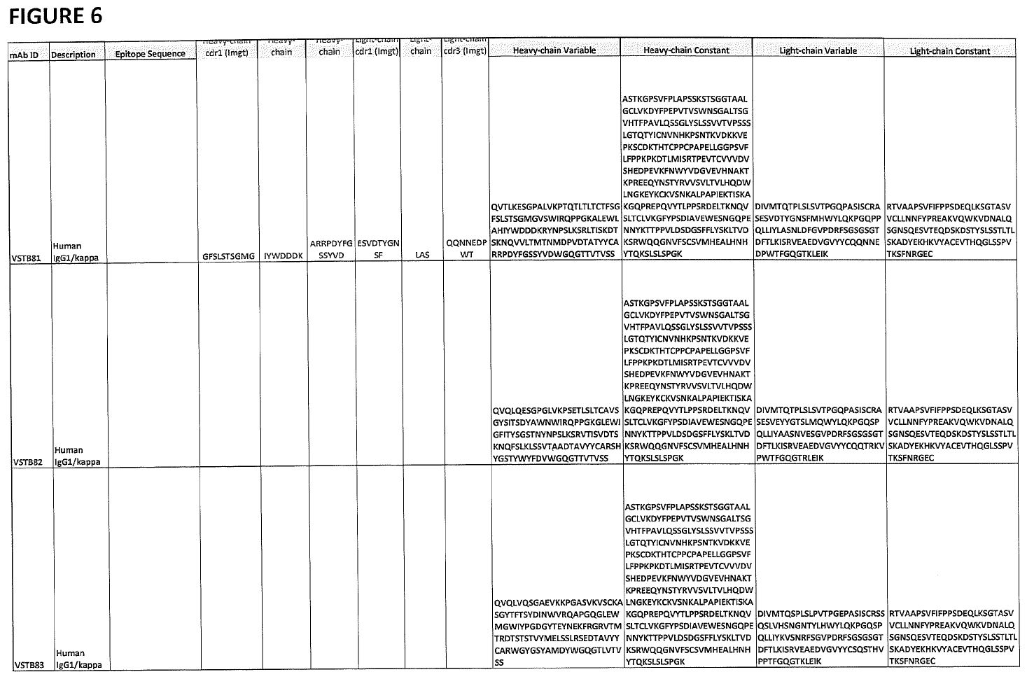

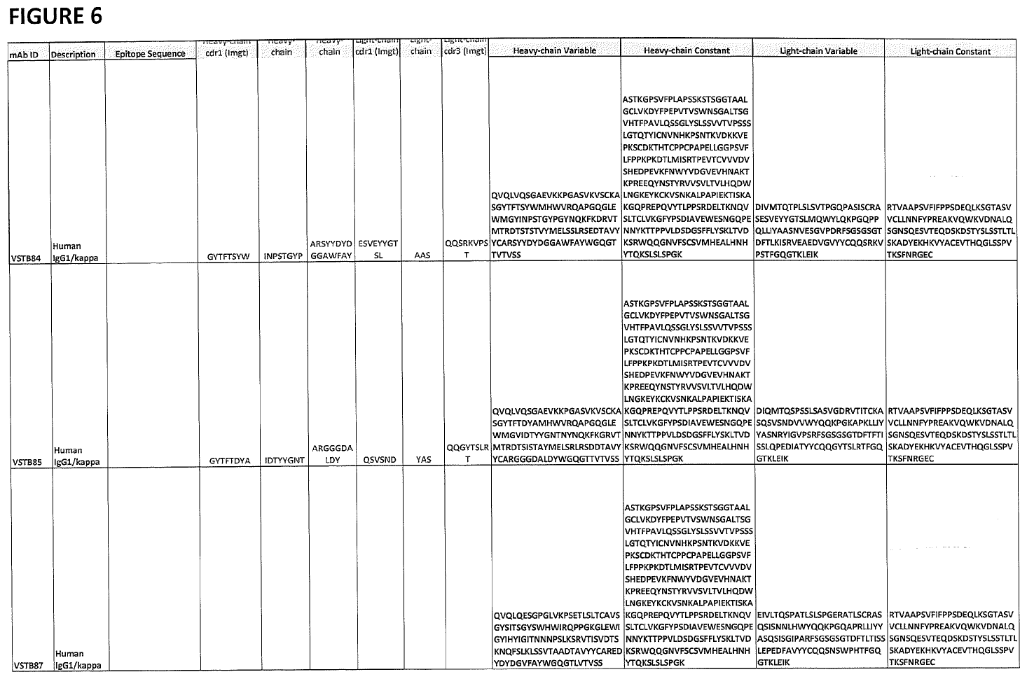

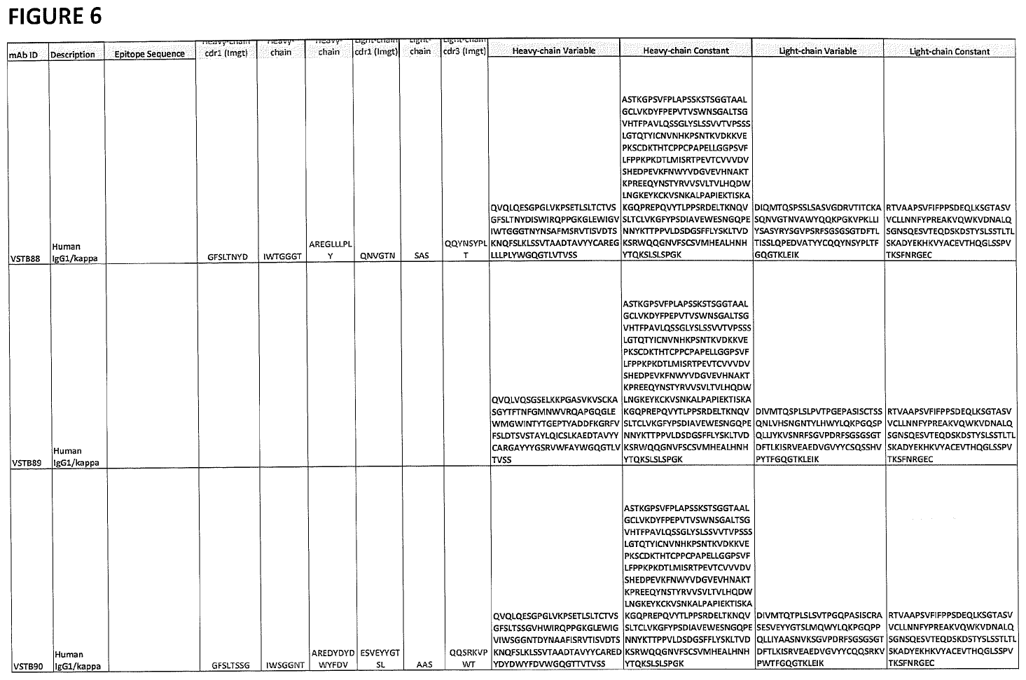

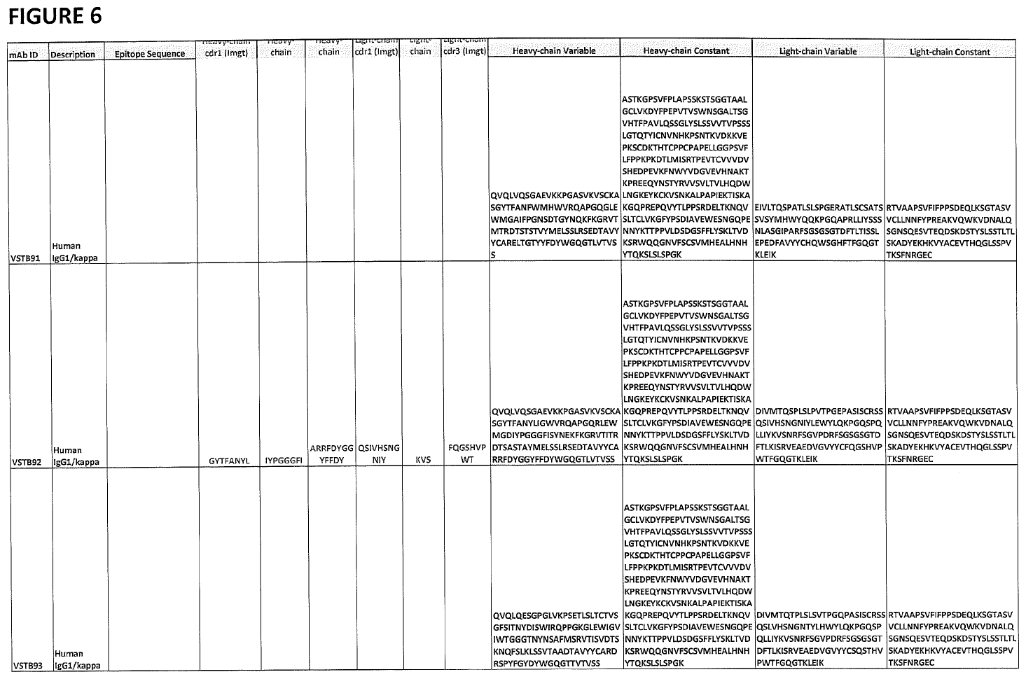

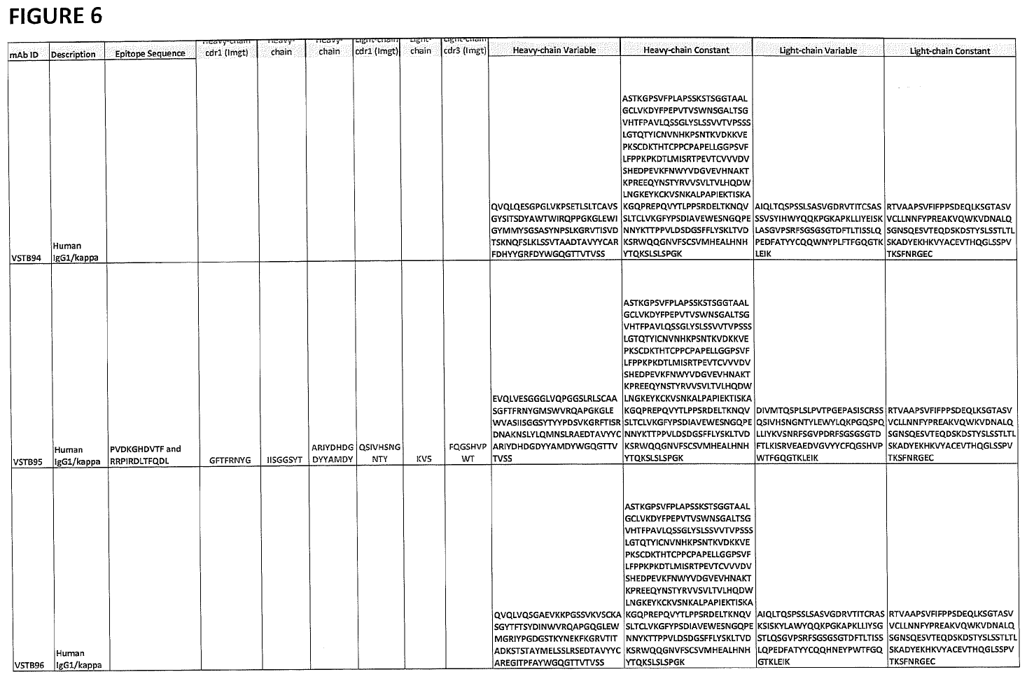

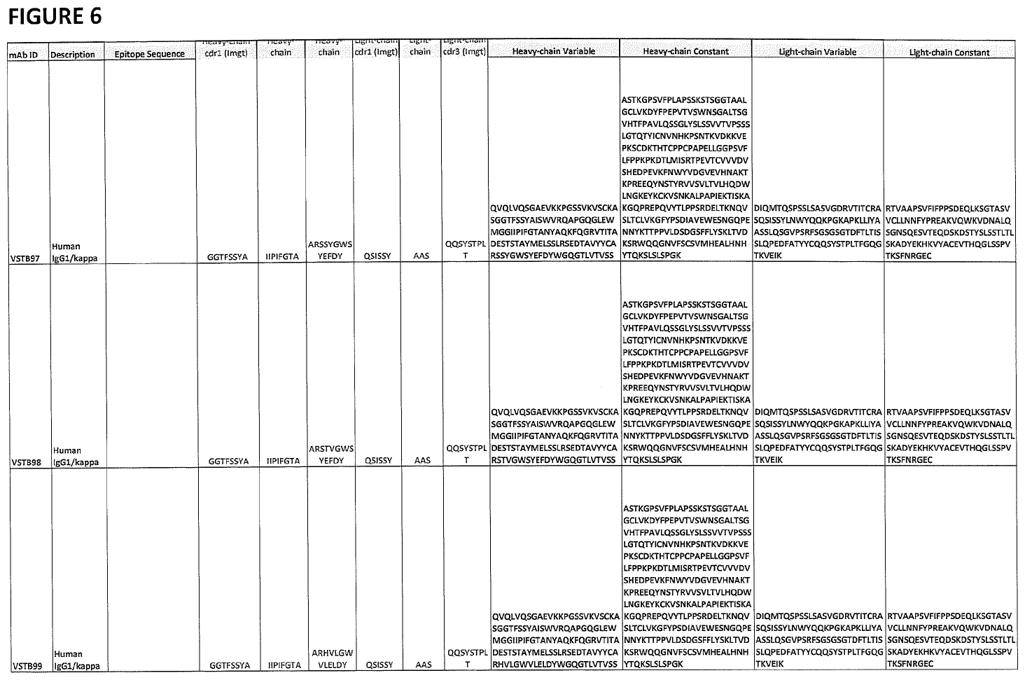

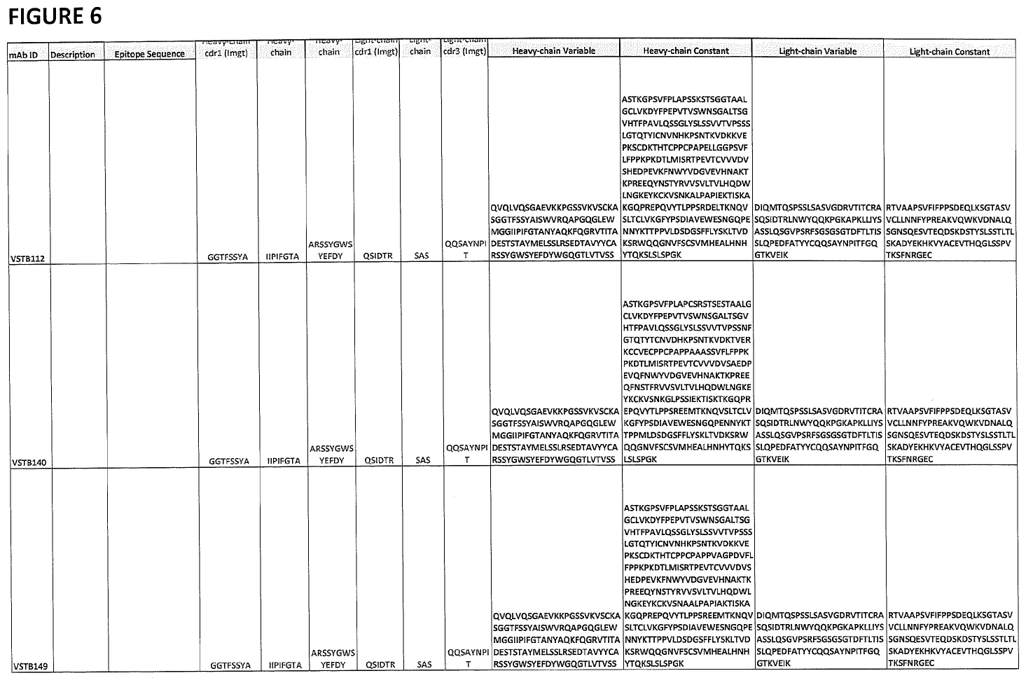

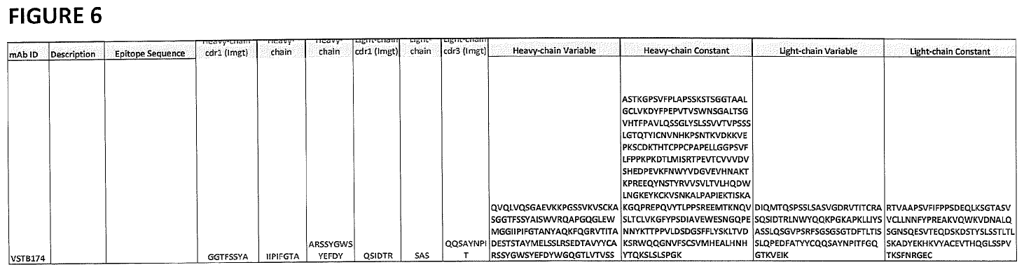

Such VISTA agonist optionally may comprise any of the VISTA agonists previously mentioned, e.g., a VISTA fusion protein, e.g., a VISTA-Ig fusion protein or more typically will comprise an agonistic anti-VISTA antibody or antibody fragment. Particularly such VISTA agonist may comprise a human VISTA fusion protein, e.g., a human VISTA-Ig fusion protein or an agonistic anti-human VISTA antibody or antibody fragment. In some exemplary embodiments the agonistic anti-VISTA or antibody fragment will comprise variable light and heavy chain polypeptide comprising the CDRs of any one of the anti-human VISTA antibodies having the sequences contained in the table in FIG. 6. In some exemplary embodiments the agonistic anti-VISTA or antibody fragment will comprise the variable light and heavy chain polypeptides of any one of the anti-human VISTA antibodies having the sequences contained in the table in FIG. 6.

VISTA agonists as disclosed herein will be used to treat or prevent IRI in any condition that comprises blood flow restoration in a tissue that suffered an anoxic event and ischemic events that result in oxygen and nutrient deprivation to tissues, and metabolic alterations and an accumulation of waste products. This includes in particular IRI that results from myocardial infarction, cardiac surgery, stroke and solid organ transplant.

Also, VISTA agonists as disclosed herein will be used to treat or prevent IRI in patients experiencing STEMI myocardial infarctions who are at high risk for IRI because in patients presenting with acute ST-elevation myocardial infarction (STEMI) the conventional therapeutic option is timely reperfusion.

VISTA agonists as disclosed herein will also specifically be used in treating or preventing IRI in patients with or at risk of Acute kidney injury (AKI) following cardiac surgery (Cardiac Surgery Associated Acute Kidney Injury (CSA-AKI)). Post-surgical AKI develops within hours to days following ischemic conditions caused by reduced blood flow to the kidneys during surgery. In particular, VISTA agonists as disclosed herein may be administered prophylactically in patients undergoing open chest cardiovascular surgery optionally with the use of cardiopulmonary bypass (CPB).

Also, VISTA agonists as disclosed herein will be used to treat or prevent IRI in patients showing signs of stroke or at risk of stroke, particularly patients suffering ischemic strokes susceptible to IRI following reperfusion after administration of tPA.

Still further VISTA agonists as disclosed herein will be used to treat or prevent IRI in transplant recipients, particularly solid organ transplants, and more particularly in patients who receive deceased donor transplants and/or those exhibiting Delayed graft function (DGF). As mentioned earlier reperfusion injury is particularly problematic in recipients who receive deceased donor transplants which may reduce graft survival, prolong hospitalization, and require dialysis.

Pharmaceutical compositions comprising at least one VISTA agonist for use in methods according to the invention can contain any pharmaceutically acceptable excipient. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. This may include e.g., aerosol, nasal, oral, transmucosal, transdermal, parenteral or enteral.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

Pharmaceutical compositions according to the invention can contain any pharmaceutically acceptable carrier. For example, the carrier may be a liquid or solid filler, diluent, excipient, solvent, or encapsulating material, or a combination thereof. In order to further describe the invention the following example is provided.

### EXAMPLES

### Example: Use of VISTA Agonist Antibody to Prevent of Cytokine Storm

Materials and Methods

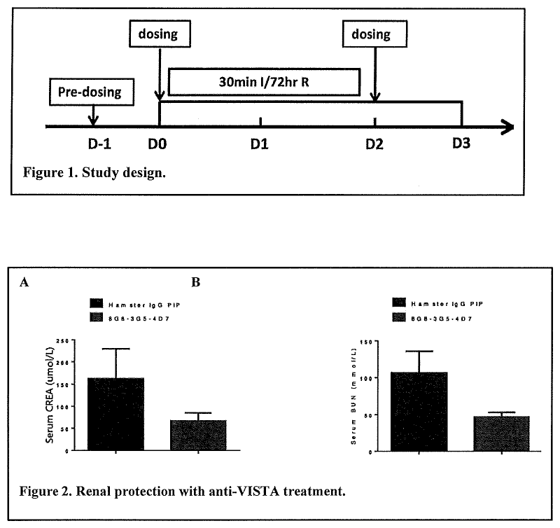

Male C57BL/6 mice (6-8 weeks old) were assigned to two groups (n=6 per group) and pretreated with an agonist anti-VISTA antibody (250 μg/treatment, i.p.) or a control antibody (250 μg/treatment, i.p.) at D-1, D0, and D2 (FIG. 1). Plasma samples were collected 72 hours after IR injury to examine the severity of renal dysfunction by measurements of plasma creatinine and blood urea nitrogen (BUN). Elevated levels of plasma creatinine and BUN are indicative of renal dysfunction and indicate that kidney glomerular filtration is impaired, i.e., lower than normal glomerular filtration rate, and that kidney damage has occurred. When the glomerular filtration rate becomes too low, patients require dialysis.

Results

Levels of plasma creatinine and BUN were significantly decreased in anti-VISTA-treated mice subjected to 30 minute renal IR compared to control-treated mice (FIG. 2A-B, n=3). Morphological assessment of hematoxylin and eosin (H&E) staining was performed by an experienced renal pathologist, who was unaware of the treatment that each animal had received.

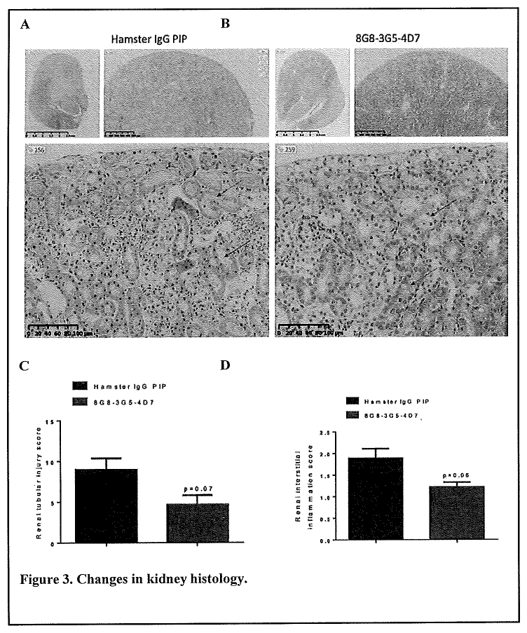

Severe tubular necrosis (FIG. 3A, red arrow) was observed in control-treated mice, and less tubular epithelial cell necrosis (FIG. 3B, red arrow) and no whole tubular necrosis was observed in anti-VISTA-treated mice.

An established grading scale of necrotic injury (0-5, renal injury score) to the proximal tubules was used for the histopathological assessment of IR-induced damage as outlined by Jablonski et al. (1983) and as described previously (Lee et al. (2007); Lee et al. (2004)). Mice treated with the agonist anti-VISTA antibody showed a decrease in the renal tubular injury score (FIG. 3C) compared with control-treated mice (FIG. 3C).

Kidney inflammation after renal IR was also assessed by the detection of inflammatory cell infiltration using H&E staining 72 hours after IR. The inflammatory infiltration score in the kidney was based on an established grading scale of inflammatory cell infiltration (0-3, inflammatory infiltration score (FIG. 3D). Mice treated with the agonist anti-VISTA antibody showed a decrease in the renal interstitial inflammation score.

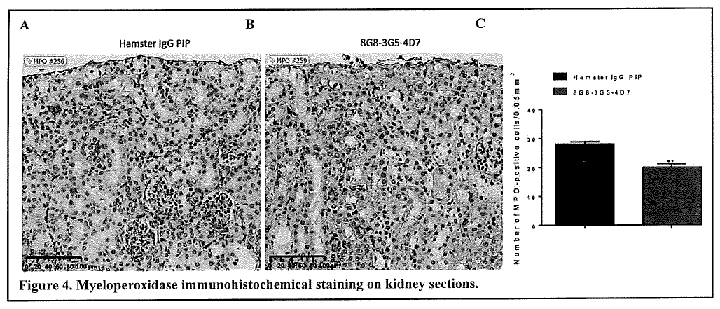

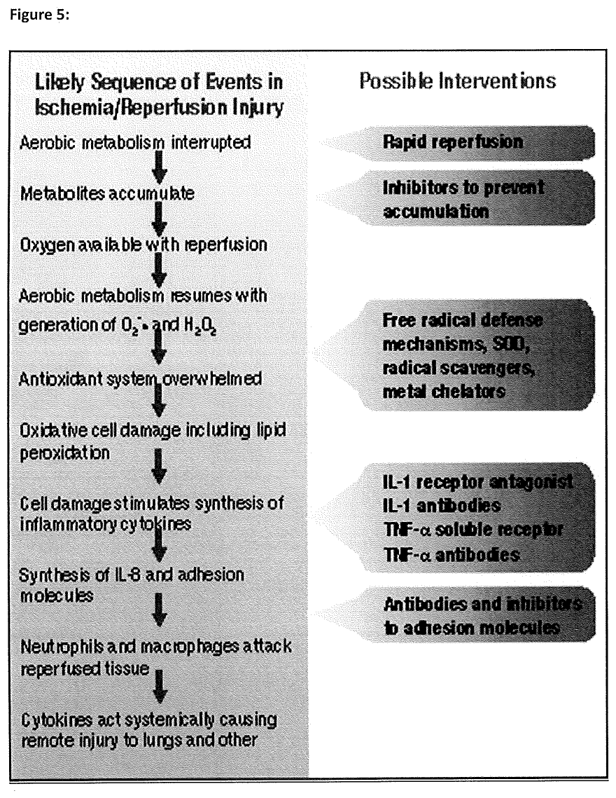

Myeloperoxidase (MPO)-positive cells were also assessed and quantified in 5 areas (0.05 mm2/area) without tissue necrosis. Many MPO-positive stained cells (FIG. 4A, red arrow) were observed in control-treated mice, especially in the glomeruli. Few MPO-positive stained cells (FIG. 4B, red arrow) were observed in the agonist anti-VISTA antibody-treated mice, and no MPO-positive stained cells were observed in the glomeruli. Results were quantified and expressed as the number of positive MPO-positive cells/0.05 mm² (FIG. 4C).

These results demonstrate that VISTA agonists may be used therapeutically or prophylactically to treat or prevent IRI, and conditions associated therewith including transplant and other IRI-associated conditions identified herein and may be used to prevent or ameliorate the adverse side effects of IRI in such conditions.

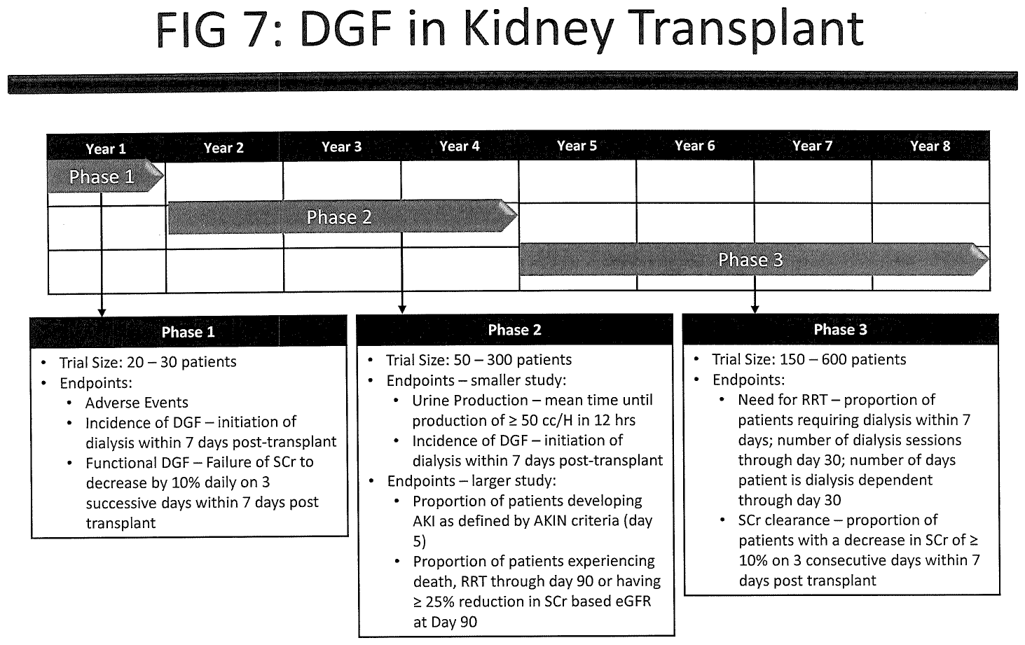

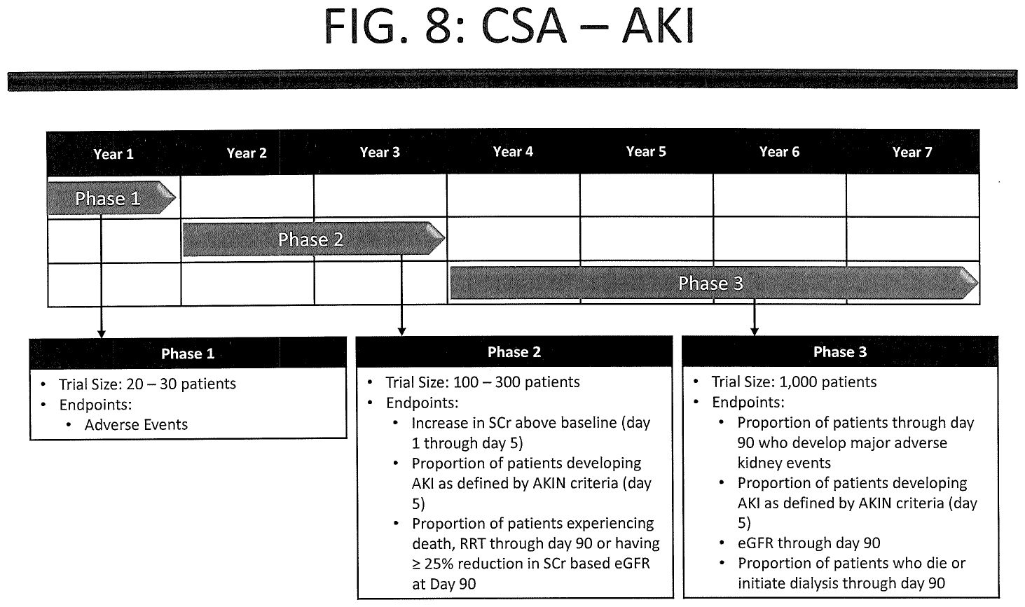

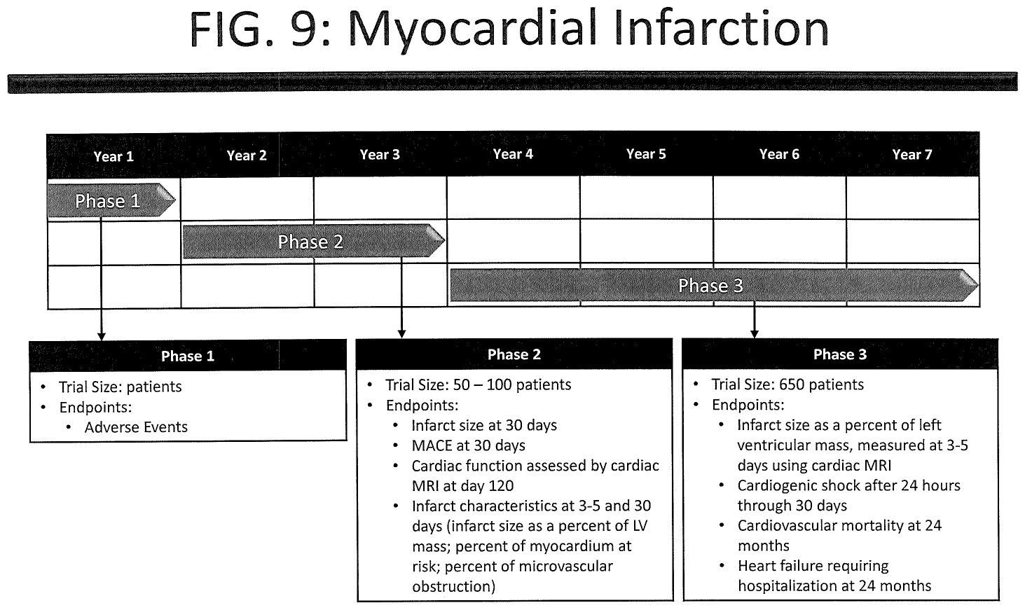

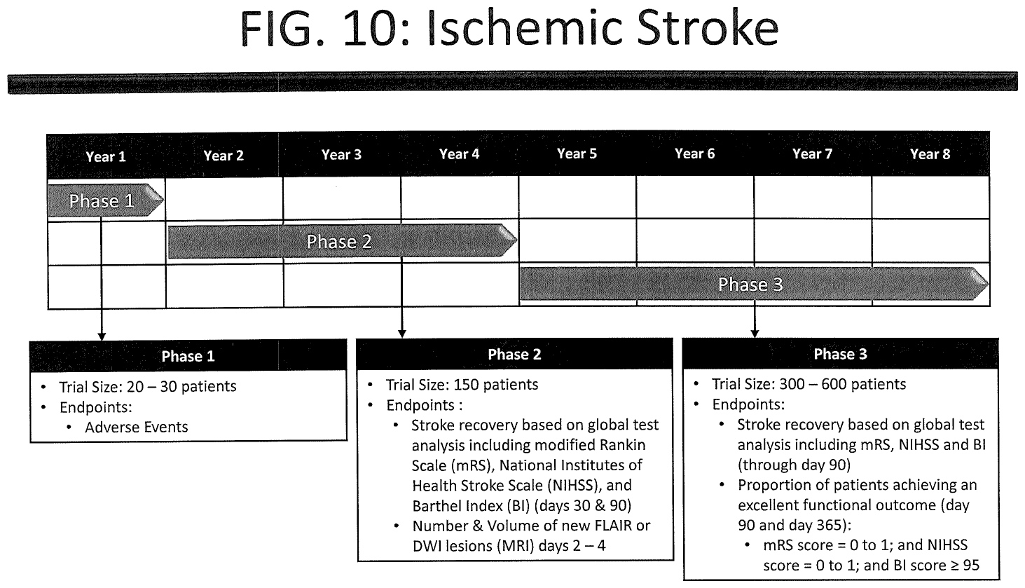

Related to the foregoing, FIGS. 7-10 respectively schematically describe proposed clinical trials relating to the use of VISTA agonists to treat DGF in kidney transplant, cardiac surgery-associated acute kidney injury, myocardial infarction and ischemic stroke.

### REFERENCES

1. Mediators Inflamm. 2017; 2017:7018393
2. Centers for Disease Control and Prevention—Heart Attack
3. European Cardiovascular Disease Statistics 2017—European Heart Network
4. AMI Trends: Incidence, Detection, Treatment. Truven Health Analytics
5. Society of Thoracic Surgeons—STS Adult Cardiac Surgery Database. Executive Summary—Harvest 4 2018
6. European Cardiovascular Disease Statistics 2017—European Heart Network
7. Ann Thorac Surg. 2012; 93: 337-47
8. Cardiorenal Med. 2013; 3: 178-199
9. Circulation. 2019; 139: e56-e528
10. European Cardiovascular Disease Statistics 2017—European Heart Network
11. Target: Stroke. American Heart Association
12. Nephron 2018; 140:94-98
13. J Transplant. 2013; 2013: 521369
14. Human Immunol. 2017: 78: 9-15
15. Global Observatory on Donation and Transplantation

Human IgG2 Heavy Constant Polypeptide
and Nucleic Acid (cDNA) Sequences
1.
ASTKGPSVFPLAPCSRSTSESTAALGCLVKDYFPEPVTVS
WNSGALTSGVHTFPAVLQSSGLYSLSSVVTVPSSNFGTQT
YTCNVDHKPSNTKVDKTVERKCCVECPPCPAPPVAGPSVF
LFPPKPKDTLMISRTPEVTCVVVDVSHEDPEVQFNWYVDG
VEVHNAKTKPREEQFNSTFRVVSVLTVVHQDWLNGKEYKC
KVSNKGLPAPIEKTISKTKGQPREPQVYTLPPSREEMTKN
QVSLTCLVKGFYPSDISVEWESNGQPENNYKTTPPMLDSD
GSFFLYSKLTVDKSRWQQGNVFSCSVMHEALHNHYTQKSL
SLSPELQLEESCAEAQDGELDGLWTTITIFITLFLLSVCY
SATITFFKVKWIFSSVVDLKQTIVPDYRNMIRQGA
2.
ASTKGPSVFPLAPCSRSTSESTAALGCLVKDYFPEPVTVS
WNSGALTSGVHTFPAVLQSSGLYSLSSVVTVPSSNFGTQT
YTCNVDHKPSNTKVDKTVERKCCVECPPCPAPPVAGPSVF
LFPPKPKDTLMISRTPEVTCVVVDVSHEDPEVQFNWYVDG
VEVHNAKTKPREEQFNSTFRVVSVLTVVHQDWLNGKEYKC
KVSNKGLPAPIEKTISKTKGQPREPQVYTLPPSREEMTKN
QVSLTCLVKGFYPSDISVEWESNGQPENNYKTTPPMLDSD
GSFFLYSKLTVDKSRWQQGNVFSCSVMHEALHNHYTQKSL
SLSPGK
3. ENST00000390545.3
4. ENST00000641095.1
5. ENST00000641095.1
6. ENST00000390545.3

1. A method of treating and/or preventing ischemic reperfusion injury (IRI) and/or adverse side effects associated with IRI in a subject in need thereof by administering a therapeutically or prophylactically effective amount of a VISTA agonist.

2. The method of claim 1, the subject is or has received a solid organ transplant, optionally from a deceased donor, and the VISTA agonist is administered prior, during or after transplant.

3. The method of claim 2, wherein the solid organ is optionally selected from a kidney, liver, heart, lung, intestine, and aorta.

4. The method of claim 2 or 3, wherein the administration of the VISA agonist, optionally an agonist anti-VISTA antibody, prior or after IR injury, prevents or mitigates the effects of solid organ injury, e.g., kidney injury and protects the solid organ, e.g., a kidney.

5. The method of any of the foregoing claims wherein the subject has or is to receive a solid organ transplant and the treatment prevents or ameliorates delayed graft function (DGF).

6. The method of any of the foregoing claims wherein the VISTA agonist prevents or treats an injury to an organ caused by ischemia, followed by reperfusion.

7. The method of any of the foregoing claims wherein the VISTA agonist prevents or treats IRI caused by one or more of the following: surgery, optionally surgery involving major organs, including but not limited to, the kidney, liver, heart, lung, intestine, and aorta, coronary bypass, major vascular repair, liver resection, and transplantation of one or more of the kidney, liver, heart, lung and aorta.

8. The method of any of the foregoing claims wherein the VISTA agonist prevents or treats IRI caused by cardiopulmonary bypass during surgery, stroke, liver ischemia, kidney ischemia, aortic occlusion, myocardial occlusion, cardiac arrest, shock and trauma.

9. The method of any of the foregoing claims wherein the VISTA agonist prevents or treats IRI caused by ischemic reperfusion injury (IRI) associated with myocardial infarction, cardiac surgery, stroke, solid organ transplant, post-surgery acute kidney injury, cardiopulmonary bypass surgery, liver ischemia, kidney ischemia, aortic occlusion, myocardial occlusion, cardiac arrest, shock and trauma.

10. The method of any of the foregoing claims wherein the VISTA agonist is an agonist anti-VISTA antibody which prevents or treats IRI caused by ischemia followed by reperfusion caused by the return of blood supply to a tissue (reperfusion) after a period of lack of oxygen (ischemia), optionally wherein said antibody comprises a human IgG2 Fc or human IgG2 constant region, further optionally wherein said human IgG2 Fc or human IgG2 constant region is not FcR impaired, i.e., it binds to human FcRs including CD32 or CD32A which are bound by naturally occurring human IgG2 Fc and human IgG2 polypeptides, further optionally wherein said human IgG2 comprises the native (unmodified) human IgG2 constant region or comprises the amino acid sequence of or encoded by any of the sequences (1) to (6) disclosed herein.

11. The method of any of the foregoing claims wherein the VISTA agonist, optionally an agonist anti-VISTA antibody, prevents or treats IRI caused by post-operative organ dysfunction due to IR injury, e.g., in a subject undergoing a major surgical procedure, optionally cardiac surgery, liver transplantation, liver resection, renal transplantation, lung transplantation, aortic surgery, or major vascular repair.

12. The method of any of the foregoing claims wherein the VISTA agonist, optionally an agonist anti-VISTA antibody, prevents or treats remote organ injury associated with IRI, optionally in a solid organ transplant recipient, further optionally a kidney transplant recipient who develops kidney injury due to renal IR and further develops one or more of liver, intestine, and lung dysfunction and/or an inflammatory state suggesting the onset of sepsis.

13. The method of any of the foregoing claims wherein the VISTA agonist, optionally an agonist anti-VISTA antibody, prevents or treats acute kidney injury (AKI), optionally associated with cardiac surgery.

14. The method of any of the foregoing claims wherein the VISTA agonist, optionally an agonist anti-VISTA antibody, and prevents or treats ischemic AKI associated with a major surgical procedure, optionally one involving the kidney, liver, heart or aorta.

15. The method of any of the foregoing claims wherein the VISTA agonist, optionally an agonist anti-VISTA antibody, and prevents or treats IRI associated multi-organ dysfunction and/or systemic inflammation.

16. The method of any of the foregoing claims wherein the VISTA agonist, optionally an agonist anti-VISTA antibody, is administered prior, during and/or after a major surgery and protects against IR injury.

17. The method of any of the foregoing claims wherein the VISTA agonist, optionally an agonist anti-VISTA antibody, is administered prior, during and/or after a surgery involving major organs, including but not limited to, the kidney, liver, heart, lung, intestine, and aorta, coronary bypass, major vascular repair, liver resection, and transplantation of the kidney, liver, and lung and protects against IRI injury.

18. The method of any of the foregoing claims wherein the VISTA agonist, optionally an agonist anti-VISTA antibody, is administered prior to a surgery including cardiopulmonary bypass.

19. The method of any of the foregoing claims wherein the VISTA agonist, optionally an agonist anti-VISTA antibody, is administered to a subject diagnosed with or showing signs of stroke, liver ischemia, kidney ischemia, aortic occlusion, myocardial occlusion, cardiac arrest, shock or trauma and such administration protects against and/or inhibits further IR injury.

20. The method of any of the foregoing claims wherein the administered VISTA agonist comprises a VISTA fusion protein, e.g., a VISTA-Ig fusion protein or comprises an agonistic anti-VISTA antibody or antibody fragment.

21. The method of any of the foregoing claims wherein the administered VISTA agonist comprises a human VISTA fusion protein, e.g., a human VISTA-Ig fusion protein or an agonistic anti-human VISTA antibody or antibody fragment.

22. The method of any of the foregoing claims wherein the administered VISTA agonist comprises an agonistic anti-VISTA or antibody fragment which comprises a variable light and heavy chain polypeptide comprising the CDRs of any one of the anti-human VISTA antibodies having the sequences contained in the table in FIG. 6.

23. The method of any of the foregoing claims wherein the administered VISTA agonist comprises an agonistic anti-VISTA or antibody fragment which comprises the variable light and heavy chain polypeptides of any one of the anti-human VISTA antibodies having the sequences contained in the table in FIG. 6.

24. The method of any of the foregoing claims wherein the administered VISTA agonist is used to treat or prevent IRI in any condition that comprises blood flow restoration in a tissue that suffered an anoxic event and ischemic events that result in oxygen and nutrient deprivation to tissues, and metabolic alterations and an accumulation of waste products, optionally myocardial infarction, cardiac surgery, stroke or solid organ transplant.

25. The method of any of the foregoing claims wherein the administered VISTA agonist is used to treat or prevent IRI in patients experiencing STEMI myocardial infarctions.

26. The method of any of the foregoing claims wherein the administered VISTA agonist is used to treat or prevent IRI in patients with or at risk of Acute kidney injury (AKI) following cardiac surgery (Cardiac Surgery Associated Acute Kidney Injury (CSA-AKI)).

27. The method of any of the foregoing claims wherein the administered VISTA agonist is used to treat or prevent IRI in patients showing signs of stroke or at risk of stroke, particularly a patient suffering ischemic strokes susceptible to IRI following reperfusion after administration of tPA.

28. The method of any of the foregoing claims wherein the administered VISTA agonist is used to treat or prevent IRI in transplant recipients, particularly solid organ transplants, and more particularly in patients who receive deceased donor transplants and/or those exhibiting Delayed graft function (DGF) in order to promote graft survival or any combination of the foregoing.

29. The method of any of the foregoing claims wherein the VISTA agonist comprises an agonist anti-VISTA antibody or agonistic anti-VISTA antibody fragment or an agonistic VSIG3 fusion protein or an agonistic anti-VSIG3 antibody or agonistic anti-VSIG3 antibody fragment or an agonistic PSGL1 antibody, antibody fragment or PSGL1 fusion protein or any combination of the foregoing.

30. The method of any of the foregoing claims wherein the VISTA agonist decreases the levels of at least one of LPS-induced IL-12p40, IL-6, CXCL2 and TNF.

31. The method of any of the foregoing claims wherein the VISTA agonist increases the expression of mediators involved in macrophage tolerance induction, wherein said mediators optionally include at least one of IRG1, miR221, A20, and IL-10 and/or increases the expression of anti-inflammatory transcription factors which drive an anti-inflammatory profile optionally including at least one of IRF5, IRF8, and NFKB1.

32. The method of any of the foregoing claims wherein the VISTA agonist (i) reduces the level of CXCR2 and/or CXCL10; (ii) reduces neutrophil/lymphocyte ratios, (iii) reduces FcgRIII levels or a combination of the foregoing.

33. The method of any of the foregoing claims wherein the VISTA agonist increases the expression of mediators involved in macrophage tolerance induction, wherein said mediators optionally include at least one of IRG1, miR221, A20, and IL-10 and/or increases the expression of anti-inflammatory transcription factors which drive an anti-inflammatory profile optionally including at least one of IRF5, IRF8, and NFKB1.

34. The method of any of the foregoing claims, which includes the administration of another active, optionally selected from a PD-1 agonist, a CTLA-4 agonist, a TNF antagonist optionally an anti-TNF antibody or TNF-receptor fusion such as Embrel, an IL-6 antagonist such as an anti-IL-6 or anti-IL-6R antibody, a corticosteroid or other anti-inflammatory agent or any combination of the foregoing.

35. The method of any of the foregoing claims, wherein the agonistic anti-VISTA antibody or antibody fragment specifically binds to human VISTA.

36. The method of claim 35, wherein the agonistic anti-VISTA antibody or antibody fragment comprises a variable light chain and a variable heavy chain polypeptide comprising the same CDRs any one of the antibodies having the sequences contained in the table in FIG. 6.

37. The method of any of the foregoing claims, wherein the agonistic anti-VISTA antibody or antibody fragment comprises a variable light chain and a variable heavy chain polypeptide comprising the same CDRs any one of the antibodies having the sequences contained in the table in FIG. 6 and the variable light chain and the variable heavy chain polypeptide of said antibody or antibody fragment respectively each possess at least 90% sequence identity to the variable light chain and the variable heavy chain polypeptides of the same anti-human VISTA antibody having the sequences contained in the table in FIG. 6.

38. The method of any of the foregoing claims, wherein the agonistic anti-VISTA antibody or antibody fragment comprises a variable light chain and a variable heavy chain polypeptide comprising the same sequences as any one of the anti-human VISTA antibodies having the sequences contained in the table in FIG. 6.

39. The method of any of the foregoing claims, wherein the VISTA agonist comprises a human VISTA fusion polypeptide, e.g., a human VISTA-Ig fusion protein and/or a human VSIG3 fusion polypeptide, e.g., a human VSIG3-Ig fusion protein or any combination of the foregoing.

40. The method of any of the foregoing claims wherein the VISTA agonist comprises a human Fc region, e.g., a human IgG1, IgG2, IgG3 or IgG4 Fc region.

41. The method of any of the foregoing claims wherein the VISTA agonist comprises a human IgG2 Fc region.

42. The method of any of the foregoing claims wherein the VISTA agonist comprises a human Fc region, e.g., a human IgG1, IgG2, IgG3 or IgG4 Fc region, that has been mutated to alter (increase or decrease) at least one effector function, e.g., FcR binding, complement binding, glycosylation, or ADCC.

43. The method of any of the foregoing claims wherein the VISTA agonist comprises a human IgG2 Fc region which binds to all or at least one Fc receptor bound by an endogenous human IgG2 Fc region.

44. The method of any of the previous claims, wherein the levels of at least one cytokine or anti-inflammatory molecule or proinflammatory molecule, e.g., CXCL10, CXCR2, IL-6, CRP, gamma interferon, IL-1, TNF, IFN-γ, IL-2, IL-17, CCL5/Rantes, CCL3/MIP-1alpha, and CXCL11/I-TAC in the patient are detected prior to treatment.

45. The method of claim 44, wherein the levels of said cytokine or proinflammatory molecule in the patient are detected and confirmed to be aberrant prior to treatment.

46. The method of any of the previous claims, wherein the levels of VISTA in the patient are detected prior to and/or after treatment.

47. The method of any of the previous claims, wherein the levels of at least one of CXCL10, CXCR2, IL-6, CRP, IFN-γ, IL-2, IL-17, CCL5/Rantes, CCL3/MIP-1alpha, and CXCL11/I-TAC in the patient are detected prior to and/or after treatment.

48. The method of claim 47, wherein the levels of IL-6 and/or CRP and/or any of IFN-γ, IL-2, IL-17, CCL5/Rantes, CCL3/MIP-1alpha, and CXCL11/I-TAC in the patient are detected and confirmed to be elevated prior to treatment.

49. The method of any of the previous claims, wherein the VISTA agonist is administered at a dose ranging from 0.01-5000 mg, 1-1000 mg, 1-500 mg, 5 mg-50 mg or about 1-25 mg.

50. The method of any of the previous claims, wherein the VISTA agonist is administered every 2 or 3 days, biweekly, weekly, every 2 or 3 weeks, or every 4 weeks intravenously or via subcutaneous injection.

51. The method of any of the previous claims, wherein the patient receives another anti-inflammatory treatment, optionally one or more of corticosteroids; inhaled nitric oxide (NO); extracorporeal membrane oxygenation (venovenous or venoarterial) or another immunosuppressive agent, optionally thymoglobulin, basiliximab, mycophenolate mofetil, tacrolimus, an anti-CD20 mAb such as rituximab, or a corticosteroid.

52. The method of any of the previous claims, wherein the patient is additionally treated with another biologic, e.g. another antibody that targets a checkpoint protein such as PD-1, PD-L1, PD-L2, CTLA-4 or an IL-6 antagonist.

53. The method of any of the previous claims wherein the anti-VISTA antibody or antibody fragment contains an Fc region that has been modified to alter effector function, half-life, proteolysis, and/or glycosylation.

54. The method of any of the previous claims wherein the anti-VISTA antibody is selected from a humanized, single chain, or chimeric antibody and the antibody fragment is selected from a Fab, Fab′, F(ab′)2, Fv, or scFv.

55. The method of any of the previous claims wherein the patient aberrantly expresses one or more biomarkers correlated with the onset or increased risk of stroke or myocardial infarction, wherein said biomarkers optionally include low-density lipoprotein-cholesterol, hemoglobin A1c (HgA1c), C-reactive protein, lipoprotein-associated phospholipase A2, urinary albumin excretion, natriuretic peptides, glial fibrillary acidic protein, S100b, neuron-specific enolase, myelin basic protein, interleukin-6, matrix metalloproteinase (MMP)-9, D-dimer, and fibrinogen.

56. The method of any of the foregoing claims wherein the VISTA agonist is used to treat or prevent any of the following: (i) IRI that results from myocardial infarction, cardiac surgery, stroke and solid organ transplant; (ii) IRI in patients experiencing STEMI myocardial infarctions; (iii) IRI in patients with or at risk of Acute kidney injury (AKI) following cardiac surgery (Cardiac Surgery Associated Acute Kidney Injury (CSA-AKI)); (iv). IRI in patients undergoing open chest cardiovascular surgery optionally with the use of cardiopulmonary bypass (CPB); IRI in patients showing signs of stroke or at risk of stroke, particularly patients suffering ischemic strokes susceptible to IRI following reperfusion after administration of tPA; (v) IRI in transplant recipients, particularly solid organ transplants, and more particularly in patients who receive deceased donor transplants and/or those exhibiting Delayed graft function (DGF).


SEQUENCE LISTING (2 sequences)

SEQ ID NO: 1 - protein, 395 aa, Artificial Sequence
  Human IgG2 Heavy Constant Polypeptide and Nucleic Acid (cDNA)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPELQLEESCAEAQDGELDGLWTTITIFITLFLLSVCYSATITFFKVKWIFSSVVDLKQTIVPDYRNMIRQGA

SEQ ID NO: 2 - protein, 326 aa, Artificial Sequence
  Human IgG2 Heavy Constant Polypeptide and Nucleic Acid (cDNA)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK